(19) United States
(12) Patent Application Publication
(10) Pub. No.: US 2026/0047992 A1
(43) Pub. Date: Feb. 19, 2026

(54) COSMETIC COMPOSITION COMPRISING CELL CULTURE MEDIUM AND ALBUMIN, AND USE THEREOF

(71) Applicant: COSMAX, INC., Gyeonggi-Do (KR)

(72) Inventors: Dong Hoan SEO, Suwon (KR); Jin Joo PARK, Seongnam-si (KR); Youn Hwa NHO, Seongnam-si (KR); Min Ji KIM, Seongnam (KR); Chun Ho PARK, Seongnam-si (KR)

(73) Assignee: COSMAX, INC., Gyeonggi-Do (KR)

(21) Appl. No.: 18/994,277

(22) PCT Filed: May 8, 2024
(86) PCT No.: PCT/KR2024/006143

§ 371 Date: Jan. 14, 2025

(30) Foreign Application Priority Data
May 9, 2023 (KR) 10-2023-0059974
May 9, 2023 (KR) 10-2023-0059975
May 9, 2023 (KR) 10-2023-0059976
Jul. 3, 2023 (KR) 10-2023-0085994
Jul. 3, 2023 (KR) 10-2023-0089981
Jul. 3, 2023 (KR) 10-2023-0089991
Jul. 3, 2023 (KR) 10-2023-0089992

(51) Int. Cl. A61K 8/64; A61K 8/06; A61K 8/20; A61K 8/24; A61K 8/34; A61K 8/365; A61K 8/44; A61K 8/49; A61K 8/67; A61Q 19/00; A61Q 19/02; A61Q 19/08

(52) U.S. Cl. CPC A61K 8/64; A61K 8/062; A61K 8/064; A61K 8/20; A61K 8/24; A61K 8/345; A61K 8/365; A61K 8/442; A61K 8/447; A61K 8/4913; A61K 8/4946; A61K 8/675; A61Q 19/007; A61Q 19/02; A61Q 19/08; A61K 2800/56; A61K 2800/5922

(57) ABSTRACT

The present disclosure relates to a cosmetic composition including a cell culture medium and albumin as active components and use thereof. The cosmetic composition according to the present disclosure may effectively improve skin conditions such as skin moisturization, skin aging improvement, skin barrier strengthening, skin wrinkle improvement, skin elasticity enhancement, skin texture improvement, skin transparency enhancement, skin discoloration and blemish removal, skin whitening, and skin regeneration, and may alleviate inflammation by inhibiting the expression of inflammatory cytokines such as IL-6 and IL-8. Furthermore, the present disclosure relates to a composition including a cell culture medium, albumin and a solubility enhancer, and use thereof. In addition, the present disclosure relates to an oil-in-water type cosmetic composition and a water-in-oil type cosmetic composition, for skin improvement, containing a cell culture medium and albumin. Moreover, the present disclosure relates to a composite powder in which a cell culture medium and albumin are impregnated into a porous polymer, a method of preparing the composite powder, and a composition including the composite powder.

### TECHNICAL FIELD

The present disclosure relates to a cosmetic composition including a cell culture medium and albumin as active components and use thereof.

### BACKGROUND ART

The cells that make up skin cells are a type of animal cell, and representative examples are keratinocytes and fibroblasts. A general composition of a medium for culturing the cells includes DMEM (Dulbecco's Modified Eagle's Medium) and fetal bovine serum (FBS).

Serum is known to play a role in promoting growth, providing nutrients, and protecting cells from oxidation and toxins. When serum is excluded from cell culture, cell culture hardly takes place, and thus serum is recognized as an essential component for cell growth and proliferation. However, it has been recently reported that FBS may be a cause of human mad cow disease. This may threaten the safety of the tester, and since serum is collected directly from bovine fetuses, it may also be a problem in terms of animal protection. In addition, FBS is expensive, so it has the disadvantage of increasing the price of the medium.

Accordingly, there is continuous demand for the development of a composition that is safe for the body and does not contain FBS, yet exhibits excellent effects in improving skin conditions such as skin texture, skin transparency, skin moisturization, discoloration, blemishes, and skin elasticity.

In this context, the present disclosure was completed by developing a cosmetic composition that is safe for the body and exhibits excellent effects in improving skin conditions such as skin texture, skin transparency, skin moisturization, discoloration, blemishes, and skin elasticity.

Meanwhile, hypoallergenic cosmetics for skin have been developed so far to avoid causing contact dermatitis, allergic dermatitis, etc. by removing active components that may cause irritation. However, the use of active components is also required in cosmetics for those with sensitive skin due to environmental and psychological factors.

In general, when looking at the mechanism of a stimulus response or an inflammatory response, human keratinocytes or Langerhans cells produce and release various cytokines in response to external substances. These cytokines are substances that promote the activation of the immune system in the human body and are essential for causing skin irritation or the progression of local inflammatory responses. Interleukin-6 (IL-6) or interleukin-8 (IL-8) is one of the inflammatory cytokines, and when IL-6 or IL-8 is used, it is very easy to confirm whether or not the anti-inflammatory effect is implemented. Specifically, unlike other inflammatory cytokines, IL-6 or IL-8 is an inflammatory gene that exists in human skin keratinocytes, and when irradiated with ultraviolet rays, phosphorylation of nf-kB, an inflammatory factor regulatory protein, progresses (nf-kB pathway progresses), and the expression of interleukin-6 (IL-6) or interleukin-8 (IL-8) also increases. Therefore, if the expression level of IL-6 and/or IL-8 is confirmed after irradiating human skin keratinocytes with ultraviolet rays and the expression level is reduced after ultraviolet irradiation as compared to before ultraviolet irradiation, it may be confirmed that the treated test substance promotes the self-defense mechanism of skin keratinocytes, thereby suppressing the expression of lipin-1 protein in the skin keratinocytes, thus suppressing the expression of the inflammatory genes IL-6 and/or IL-8. In other words, it may be confirmed whether the treated test substance is a substance for promoting the self-defense mechanism of skin keratinocytes.

In fact, an increase in interleukin-6 (IL-6), interleukin-1 (IL-1), and tumor necrosis factor-α (TNF-α) has been reported in irritant contact dermatitis.

In this context, the present disclosure was completed by developing a cosmetic composition that is safe for the body and exhibits excellent effects in alleviating inflammation and improving skin condition.

In addition, the present disclosure was completed by identifying a method capable of enhancing the solubility of the composition.

In addition, the present disclosure was completed by developing a technique capable of enhancing the stability of the formulation of the composition.

In addition, the present disclosure was completed by developing a technology capable of enhancing the homogenization of the composition.

### DISCLOSURE OF INVENTION

### Technical Problem

One aspect provides a cosmetic composition including a cell culture medium and albumin as active components.

Another aspect provides a topical skin composition including a cell culture medium and albumin as active components.

Another aspect provides an anti-inflammatory cosmetic composition including a cell culture medium and albumin as active components.

Another aspect provides an anti-inflammatory topical skin composition including a cell culture medium and albumin as an active component.

Another aspect provides a composition including a cell culture medium, albumin and a solubility enhancer.

Another aspect provides a method of improving the solubility of a composition including a cell culture medium and albumin, including: 1) a step of dissolving components of a cell culture medium and albumin in a solvent; and 2) a step of adding a solubility enhancer to the solution obtained in the above step and mixing the same.

Another aspect provides an oil-in-water type cosmetic composition containing a cell culture medium and albumin as active components while ensuring stability of the formulation.

Another aspect provides an oil-in-water type composition for topical skin preparation containing a cell culture medium and albumin as active components while ensuring stability of the formulation.

Another aspect provides a water-in-oil type cosmetic composition containing a cell culture medium and albumin as active components while ensuring stability of the formulation.

Another aspect provides a water-in-oil type composition for topical skin preparation containing a cell culture medium and albumin as active components while ensuring stability of the formulation.

Another aspect provides a composite powder including a cell culture medium, albumin and a porous polymer.

Another aspect provides a method of preparing a composite powder including a cell culture medium and albumin, including: 1) a step of dissolving or dispersing a cell culture medium, albumin, and a porous polymer in a solvent; and 2) a step of spray-drying the solution or dispersion.

Another aspect provides a composition including a composite powder including a cell culture medium, albumin, and a porous polymer.

### Solution to Problem

One aspect is to provide a cosmetic composition including a cell culture medium, albumin, and a solubility enhancer.

The term “cell culture medium” in the present specification contains components required by cells for cell growth and survival in vitro, or contains components that aid cell growth and survival. Specifically, the components may include vitamins, essential or non-essential amino acids, and trace elements. The medium may be a medium used for culturing cells, preferably eukaryotic cells, more preferably animal cells.

Examples of the cell culture medium include, but are not limited to, DMEM/F-12 (Dulbecco's Modified Eagle's Medium/Nutrient Mixture F-12), DMEM, MEMα (Minimum Essential Mediuma), BME (Basal Medium Eagle), GMEM (Glasgow Minimum Essential Medium), RPMI-1640 Medium, etc.

In addition, the cell culture medium of the present disclosure is a serum-free medium. Serum-free medium refers to a culture medium that does not contain serum derived from animals, including humans (for example, animal-derived serum). Animal-derived serum provides universal nutrients for the growth of cells to be cultured, but it includes unidentified trace components, making analysis difficult, making it difficult to establish reproducible testing and production processes, and cannot guarantee stability for the human body. However, the cell culture medium according to the present disclosure is a serum-free medium, which ensures stability for the human body and enables establishment of reproducible testing and production processes.

Additionally, the cell culture medium refers to a cell culture medium before culturing cells. Since it is a culture medium before culturing cells, there is no change in the components of the cell culture medium, so it may be used commercially with homogenous quality.

In an embodiment, the cell culture medium may include one or more selected from the group consisting of amino acid components, vitamin components, inorganic salt components, and other components, and may further include purified water.

The amino acid component may include one or more amino acids selected from the group consisting of glycine, L-alanine, L-valine, L-leucine, L-isoleucine, L-threonine, L-serine, L-cysteine, L-methionine, L-aspartic acid, L-asparagine, L-glutamic acid, L-glutamine, L-lysine, L-arginine, L-histidine, L-phenylalanine, L-tyrosine, L-tryptophan, L-proline, β-alanine, γ-aminobutyric acid, ornithine, citrulline, homoserine, thyroxine, and dihydroxyphenylalanine, and preferably, may include one or more amino acids selected from the group consisting of glycine, L-alanine, L-arginine, L-cysteine, L-glutamine, L-histidine, L-lysine, L-methionine, L-proline, L-serine, L-threonine, and L-valine.

The vitamin component may include one or more vitamins selected from the group consisting of biotin, calcium D-pantothenate, folic acid, niacinamide, pyridoxine hydrochloride, riboflavin, thiamine hydrochloride, vitamin B12, choline chloride, i-inositol, and ascorbic acid, and preferably, may include one or more vitamins selected from the group consisting of i-inositol, thiamine hydrochloride, niacinamide, and pyridoxine hydrochloride.

The inorganic salt component may include one or more inorganic salts selected from the group consisting of calcium chloride (CaCl₂) (anhydrous), copper sulfate pentahydrate (CuSO₄·5H₂O), ferrous sulfate heptahydrate (FeSO₄·7H₂O), magnesium chloride (anhydrous), magnesium sulfate (MgSO₄) (anhydrous), potassium chloride (KCl), sodium chloride (NaCl), disodium hydrogen phosphate (Na₂HPO₄), sodium dihydrogen phosphate monohydrate (NaH₂PO₄·H₂O), zinc sulfate heptahydrate (ZnSO₄·7H₂O), ferric nitrate nonahydrate (Fe(NO₃)₃·9H₂O), and sodium bicarbonate (NaHCO₃), and preferably, may include one or more inorganic salts selected from the group consisting of sodium chloride (NaCl), sodium bicarbonate (NaHCO₃), potassium chloride (KCl), calcium chloride (CaCl₂) (anhydrous), and sodium dihydrogen phosphate monohydrate (NaH₂PO₄·H₂O).

The other components may include one or more other components selected from the group consisting of D-glucose (dextrose), sodium pyruvate, hypoxanthine Na, thymidine, linoleic acid, lipoic acid, adenosine, cytidine, guanosine, uridine, 2′-deoxyadenosine, 2′-deoxycytidine HCl, and 2′-deoxyguanosine, and preferably, may include sodium pyruvate.

The purified water is used to dissolve the amino acids, vitamins, inorganic salts, and other components, and may be obtained through one or more distillations or purified through a filter.

In an embodiment, the cell culture medium may include glycine, L-alanine, L-arginine hydrochloride, L-cysteine hydrochloride-monohydrate, L-glutamine, L-histidine hydrochloride-monohydrate, L-lysine hydrochloride, L-methionine, L-proline, L-serine, L-threonine, L-valine, i-inositol, thiamine hydrochloride, niacinamide, pyridoxine hydrochloride, sodium chloride (NaCl), sodium bicarbonate (NaHCO₃), potassium chloride (KCl), calcium chloride (CaCl₂) (anhydrous), sodium bicarbonate monohydrate (NaH₂PO₄—H₂O), and sodium pyruvate, and may specifically be consisted of the above components.

The amino acid component is used for cell growth as a raw material for protein synthesis, and it may include 0.2 to 1.2 parts by weight of glycine, 0.08 to 0.3 parts by weight of L-alanine, 2 to 7 parts by weight of L-arginine hydrochloride, 0.2 to 0.9 parts by weight of L-cysteine hydrochloride monohydrate, 10 to 18 parts by weight of L-glutamine, 0.8 to 2.0 parts by weight of L-histidine hydrochloride monohydrate, 1.0 to 5.0 parts by weight of L-lysine hydrochloride, 0.2 to 1.2 parts by weight of L-methionine, 0.2 to 1.2 parts by weight of L-proline, 1.0 to 5.0 parts by weight of L-threonine, and 1.0 to 5.0 parts by weight of L-valine, based on 1 part by weight of serine, an amino acid included in the cell culture medium.

In an embodiment, the amino acid component and content included in the cell culture medium may be 0.5 to 1.0 parts by weight of glycine, 0.10 to 0.02 parts by weight of L-alanine, 5.0 to 6.0 parts by weight of L-arginine hydrochloride, 0.4 to 0.7 parts by weight of L-cysteine hydrochloride-monohydrate, 12 to 15 parts by weight of L-glutamine, 1.0 to 1.5 parts by weight of L-histidine hydrochloride-monohydrate, 2.0 to 4.0 parts by weight of L-lysine hydrochloride, 0.4 to 0.8 parts by weight of L-methionine, 0.4 to 0.8 parts by weight of L-proline, 1.5 to 2.5 parts by weight of L-threonine, and 1.5 to 2.5 parts by weight of L-valine. By including the amino acid component in a content within the above range, it may help cell growth and maintenance and improve formulation stability.

In the cell culture medium, the vitamin component plays a role in maintaining cell activity and may include 0.2 to 0.9 parts by weight of i-inositol, 0.04 to 0.8 parts by weight of thiamine hydrochloride, 0.04 to 0.8 parts by weight of niacinamide, and 0.04 to 0.8 parts by weight of pyridoxine hydrochloride, based on 1 part by weight of amino acid serine.

In an embodiment, the vitamin component and content included in the cell culture medium may be 0.4 to 0.7 parts by weight of i-inositol, 0.06 to 0.2 parts by weight of thiamine hydrochloride, 0.06 to 0.2 parts by weight of niacinamide, and 0.06 to 0.2 parts by weight of pyridoxine hydrochloride, based on 1 part by weight of amino acid serine. By including the vitamin component in a content within the above range, it may help maintain cellular activity.

The inorganic salt component plays a role in controlling the expression of cell function, and may include 50 to 200 parts by weight of sodium chloride (NaCl), 50 to 120 parts by weight of sodium bicarbonate (NaHCO₃), 2.0 to 20 parts by weight of potassium chloride (KCl), 1.0 to 10 parts by weight of calcium chloride (CaCl₂)) (anhydrous), and 0.5 to 5.0 parts by weight of sodium dihydrogen phosphate monohydrate (NaH₂PO₄—H₂O) based on 1 part by weight of amino acid serine.

In an embodiment, the inorganic salt component and content included in the cell culture medium may be 100 to 150 parts by weight of sodium chloride (NaCl), 80 to 100 parts by weight of sodium bicarbonate (NaHCO₃), 8 to 15 parts by weight of potassium chloride (KCl), 2.0 to 8.0 parts by weight of calcium chloride (CaCl₂)) (anhydrous), and 1.0 to 4.0 parts by weight of sodium dihydrogen phosphate monohydrate (NaH₂PO₄—H₂O) based on 1 part by weight of amino acid serine.

The other component may include components that may improve the stability, etc. of the cell culture medium, etc., and may include 0.5 to 5.0 parts by weight of sodium pyruvate based on 1 part by weight of amino acid serine.

In an embodiment, the other component and content included in the cell culture medium may be 1.0 to 4.0 parts by weight of sodium pyruvate based on 1 part by weight of amino acid serine. The other component included in a content within the above range may help improve formulation stability, etc.

The albumin included in the composition may be human serum albumin or bovine serum albumin.

The human serum albumin is serum albumin found in human blood and is the most abundant protein present in human plasma. In the present disclosure, the human serum albumin may specifically be a recombinant human serum albumin. Additionally, the bovine serum albumin is a serum albumin found in cows and is often used as a protein concentration standard in experiments.

The albumin content may be incorporated at a concentration of 10 μg/mL, 50 μg/mL, 100 μg/mL, 200 μg/mL, 300 μg/mL, 500 μg/mL, or 1000 μg/mL. Alternatively, the albumin content may be included at concentrations of 10 to 1000 μg/mL, 50 to 500 μg/mL, or 100 to 250 μg/mL.

The cosmetic composition may be for improving skin condition.

The improvement in skin condition may include one or more selected from the group consisting of skin moisturization, skin aging improvement, skin barrier strengthening, skin wrinkle improvement, skin elasticity enhancement, skin texture improvement, skin transparency enhancement, skin discoloration and blemish removal, skin whitening, and skin regeneration.

The term “skin moisturization” in the present specification may refer to any action that maintains skin moisture or prevents moisture loss.

The term “skin aging” in the present specification refers to both the tangible and intangible changes that occur in the skin with age, for example thinning of the epidermis, decrease in, the number of cells and blood vessels in the dermis, the ability to repair DNA damage, the cell turnover cycle, wound healing, skin barrier function, epidermal water retention, sweat secretion, sebum secretion, vitamin D production, physical damage defense, the ability to remove chemical substances, immune response, sensory function, and body temperature control.

The improvement in skin aging may refer to improvement in skin aging caused by exogenous factors or endogenous factors. The exogenous factors refer to various external factors, such as ultraviolet radiation (light), and endogenous factors are also referred to as chronological factors, and mainly refer to factors caused by the passage of time. In other words, the skin aging specifically includes not only premature aging symptoms induced by external stimuli such as ultraviolet rays, pollution, cigarette smoke, chemicals, etc., but also is a concept that includes the natural aging phenomenon that occurs as skin cell proliferation decreases with age, and includes wrinkles, loss of elasticity, skin sagging, and dryness. In addition, wrinkles include stimulation caused by changes in internal and external factors that change the components that make up skin tissue, causing wrinkles.

The aging may be photoaging. The term “photoaging” is a phenomenon caused by external environmental factors, the most representative of which is ultraviolet rays. Ultraviolet rays cause damage to biological components such as activation of proteolytic enzymes, chain cleavage of matrix proteins, and abnormal cross-linking, etc., and repetition of this mechanism causes skin aging that is evident in appearance.

The term “skin barrier strengthening” in the present specification may refer to any action that enhance the function of the skin barrier, which is located on the outermost layer of the skin and prevents moisture and nutrition loss.

The term “wrinkle” in the present specification refers to a state in which the skin loses its elasticity and becomes loose, for example, folding of the skin. The term “prevention or improvement of skin wrinkles” may refer to any action that prevents or improves wrinkles by inhibiting the expression of factors related to wrinkles, or increases the total amount of collagen.

The term “skin transparency” in the present specification refers to the ratio of transmitted light to incident light at the skin surface. The incident light is scattered on the skin surface and some of it is diffused, while the remaining light transmitted into the skin, and the transmitted light is reflected back out from inside the skin. As the amount of light reflected from within the skin (diffuse reflection) increases, skin transparency increases.

The term “skin elasticity enhancement” in the present specification refers to alleviating the degree of sagging or loosening of the skin.

The term “skin texture” in the present specification refers to a three-dimensional microstructure formed by fine lines on the skin surface. Aside from elasticity, wrinkles, and moisturization, etc. it is an indicator of skin condition, and more specifically, skin texture becomes a reliable indicator of aging that may detect aging before it begins. In addition, skin texture is not simply the condition of the rough and smooth skin surface, but rather reflects the internal condition of the skin, the visible skin texture of the skin surface is only a portion of the skin's overall structure that is revealed, and damaged skin texture is a reflection of changes within the skin.

The term “skin regeneration” in the present specification may refer to any action that replenishes a portion of the skin that has been lost or promotes the proliferation of skin cells. Specifically, skin regeneration may refer to the regeneration of skin tissue at the site of a skin wound.

The term “skin whitening” in the present specification may refer to not only brightening the skin tone by inhibiting the synthesis of melanin pigment, but also improving skin hyperpigmentation such as discoloration or freckles, etc. caused by ultraviolet rays, hormones, or genetics.

The term “blemish” in the present specification is thought to be caused by a complex interplay of various factors, such as cloudiness or shade due to thickening of the stratum corneum accompanying aging, decreased redness of the skin, deposition of pigments, decreased elasticity of the skin, yellowing of the skin, and contamination with sweat or sebum, etc.

The cosmetic composition may have enhanced skin condition improvement efficiency, and specifically, may have enhanced skin condition improvement efficiency compared to a composition that does not include a cell culture medium and/or albumin as active components.

The cell culture medium and albumin, or a cosmetic composition including the same, may be anti-inflammatory.

The term “anti-inflammatory” in the present specification refers to the process of causing inflammation or the property or efficacy of suppressing it, and the anti-inflammatory may refer to one or more selected from the group consisting of 1) improving or alleviating skin inflammation, 2) soothing the skin; and 3) improving or preventing dermatitis.

The above skin soothing refers to calming or alleviating irritation, fever, and/or pain, etc. of irritated skin.

The dermatitis may include one or more selected from the group consisting of atopic dermatitis, allergic dermatitis, eczema, contact dermatitis, erythema, and psoriasis.

The cell culture medium and albumin, or a cosmetic composition including the same, may inhibit the production, activity, or expression of inflammatory cytokines.

The inflammatory cytokine may include one or more selected from the group consisting of TNF-α, IL-1β, IL-4, IL-6, IL-8, IL-9, and IL-13.

The cosmetic composition may be for improving skin condition by alleviating inflammation.

The cosmetic composition may have enhanced skin condition improvement efficiency by alleviating inflammation, and specifically, may have enhanced skin condition improvement efficiency compared to a composition that does not include a cell culture medium and/or albumin as active components.

Specifically, the skin condition improvement efficiency of the cosmetic composition may be enhanced by 10% or more, 30% or more, 50% or more, 70% or more, 100% or more, 150% or more, 200% or more, or 300% or more, compared to a composition that does not include a cell culture medium and/or albumin as active components, and specifically, may be enhanced by 10% to 300%, 10% to 200%, 10% to 150%, 10% to 100%, 10% to 70%, 10% to 50%, 30% to 300%, 30% to 200%, 30% to 150%, 30% to 100%, 30% to 70%, 30% to 50%, 50% to 300%, 50% to 200%, 50% to 150%, 50% to 100%, or 50% to 70%.

The formulation of the cosmetic composition is not particularly limited, and for example, may have any one formulation selected from among toners, lotions, essences, creams, packs, foundations, patches, hair tonics, microneedle patches, and makeup bases, but may not be limited thereto.

The cosmetic composition may add a substance arbitrarily selected according to the formulation or intended use of the cosmetic, but may not be limited thereto. For example, purified water, oil, surfactants, moisturizers, higher alcohols, thickeners, chelating agents, pigments, fatty acids, antioxidants, preservatives, waxes, pH adjusters, fragrances, etc. may be added, but may not be limited thereto.

When the formulation of the cosmetic composition is a paste, cream or gel, it may include, animal oil, vegetable oil, wax, paraffin, starch, tragacanth, cellulose derivatives, polyethylene glycol, silicone, bentonite, silica, talc or zinc oxide, etc. as a carrier component, but may not be limited thereto.

When the formulation of the cosmetic composition is a powder or spray, it may include lactose, talc, silica, aluminum hydroxide, calcium silicate or polyamide powder as a carrier component, and in particular, if it is a spray, may further include a propellant such as chlorofluorohydrocarbon, propane/butane or dimethyl ether, but may not be limited thereto.

When the formulation of the cosmetic composition is a solution or an emulsion, it may include a solvent, a solubilizer or an emulsifier as a carrier component, and may include, for example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan, but may not be limited thereto.

When the formulation of the cosmetic composition is a suspension, it may include a liquid diluent such as water, ethanol or propylene glycol as a carrier component, suspending agents such as ethoxylated isostearyl alcohol, polyoxyethylene sorbitol ester and polyoxyethylene sorbitan ester, microcrystalline cellulose, aluminum methahydroxide, bentonite, agar or tragacanth, etc., but may not be limited thereto.

When the formulation of the cosmetic composition is a surfactant-containing cleanser, the carrier component may include, an aliphatic alcohol sulfate, an aliphatic alcohol ether sulfate, a sulfosuccinic acid monoester, an isethionate, an imidazolinium derivative, methyl taurate, a sarcosinate, a fatty acid amide ether sulfate, an alkylamidobetaine, an aliphatic alcohol, a fatty acid glyceride, a fatty acid diethanolamide, a vegetable oil, a lanolin derivative, or an ethoxylated glycerol fatty acid ester, etc., but may not be limited thereto.

As an example of the cosmetic composition formulation, it may be prepared into a formulation of skincare products including softening lotion, astringent lotion, nourishing lotion, lotion, gel, cream, essence, eye essence, eye cream, nourishing cream, massage cream, clay-type pack, and mask pack; makeup products including lipstick, lip tint, lip gloss, lip pencil, eye shadow, foundation, powder, concealer, eyeliner, eye shadow, and mascara; cleansing products including eye makeup remover, makeup remover, foam cleanser, cleansing cream, cleansing oil, cleansing water, hand sanitizer, hand wash, hand scrubs, body wash, body scrubs, shaving lotion, soap, and wet wipes; body care products including body lotion, body oil, body mist, body essence, hand cream, foot cream, and wax-type hair removal agents; baby cosmetics including baby lotion, baby cream, and diaper rash cream; sun care products including sunscreen, sun stick, sun spray, and artificial tanning products; hair products including shampoo, hair conditioner, hair treatment cream, hair styling gel, foam, hair mousse, hair spray, styling lotion, styling cream, hair essence, hair dye, hair bleaching cream, and curl activator gel; nail care products including nail varnish, nail polish, cuticle remover, and cuticle cream; personal care products including toothpaste, mouthwash, oral rinse, oral films, and gum; perfume, plant extract, deodorant, and antiperspirant.

The cosmetic composition may further include a solubility enhancer.

The solubility enhancer may improve the solubility of cell culture medium components and albumin in the composition, and specifically may improve the solubility in an aqueous solution such as purified water, etc.

The solubility enhancer may include a pH adjuster.

The pH adjuster may decrease the pH of the added composition and, specifically, the pH adjuster may be an acidic substance.

The pH adjuster may be added in an amount appropriate for adjusting the pH to be obtained in the composition, and specifically may be added to adjust the pH of the entire composition to within a range of 3.0 to 9.0.

In an embodiment, the composition may have a pH of 3.0 to 9.0, and more specifically, a pH in the range of 6.0 to 8.0, and even more specifically, a pH in the range of 3.0 to 9.0, 3.0 to 8.5, 3.0 to 8.0, 3.0 to 7.8, 3.0 to 7.6, 3.0 to 7.5, 3.5 to 9.0, 3.5 to 8.5, 3.5 to 8.0, 3.5 to 7.8, 3.5 to 7.6, 3.5 to 7.5, 4.0 to 9.0, 4.0 to 8.5, 4.0 to 8.0, 4.0 to 7.8, 4.0 to 7.6, 4.0 to 7.5, 4.5 to 9.0, 4.5 to 8.5, 4.5 to 8.0, 4.5 to 7.8, 4.5 to 7.6, 4.5 to 7.5, 5.0 to 9.0, 5.0 to 8.5, 5.0 to 8.0, 5.0 to 7.8, 5.0 to 7.6, 5.0 to 7.5, 5.5 to 9.0, 5.5 to 8.5, 5.5 to 8.0, 5.5 to 7.8, 5.5 to 7.6, 5.5 to 7.5, 6.0 to 9.0, 6.0 to 8.5, 6.0 to 8.0, 6.0 to 7.8, 6.0 to 7.6, 6.0 to 7.5, 6.5 to 9.0, 6.5 to 8.5, 6.5 to 8.0, 6.5 to 7.8, 6.5 to 7.6, 6.5 to 7.5, 7.0 to 9.0, 7.0 to 8.5, 7.0 to 8.0, 7.0 to 7.8, 7.0 to 7.6, 7.0 to 7.5, 7.2 to 9.0, 7.2 to 8.5, 7.2 to 8.0, 7.2 to 7.8, 7.2 to 7.6, 7.2 to 7.5, 7.4 to 9.0, 7.4 to 8.5, 7.4 to 8.0, 7.4 to 7.8, 7.4 to 7.6, 7.4 to 7.5, 7.5 to 9.0, 7.5 to 8.5, 7.5 to 8.0, 7.5 to 7.8, or 7.5 to 7.6.

The pH adjuster may include an organic acid and/or an inorganic acid.

The organic acid may include alpha hydroxy acid (AHA) and/or beta hydroxy acid (BHA), etc.

The term “Alpha Hydroxy Acid (AHA)” in the present specification refers to a compound in which an alcohol group or a hydroxy group is added to a carbon at the a position of a carboxylic acid. Additionally, the term “Beta Hydroxy Acid (BHA)” refers to a compound in which an alcohol group or a hydroxy group is added to a carbon at the p position of a carboxylic acid.

The pH adjuster may include one or more selected from the group consisting of citric acid, salicylic acid, glycolic acid, lactic acid, malic acid, tartaric acid, mandelic acid, poly-hydroxy acid (PHA), b-lipohydroxy acid (LHA), oxalic acid, gluconolactone, ascorbic acid, glutamic acid, D-gluconic acid, sulfonic acid, methanesulfonic acid, benzimidazole sulfonic acid, 4-aminobenzoic acid, benzoic acid, sorbic acid, phenylbenzimidazole sulfonic acid, benzylidene camphor sulfonic acid, terephthalylidene dicamphor sulfonic acid, kojic acid, hyaluronic acid, phosphoric acid, carbonic acid, hydrochloric acid, acetic acid, and formic acid, and may specifically include citric acid. The composition may be a cosmetic composition, and specifically, may be a cosmetic composition for improving skin condition.

The cosmetic composition may be an oil-in-water (O/W) type.

The cell culture medium and albumin may be included in an amount of 0.0001 to 10 wt % based on the total weight of the composition, and specifically, may be included in an amount of 0.0001 to 10 wt %, 0.0001 to 5 wt %, 0.0001 to 3 wt %, 0.0001 to 2 wt %, 0.0001 to 1 wt %, 0.001 to 10 wt %, 0.001 to 5 wt %, 0.001 to 3 wt %, 0.001 to 2 wt %, 0.001 to 1 wt %, 0.01 to 10 wt %, 0.01 to 5 wt %, 0.01 to 3 wt %, 0.01 to 2 wt %, 0.01 to 1 wt %, 0.1 to 10 wt %, 0.1 to 5 wt %, 0.1 to 3 wt %, 0.1 to 2 wt %, 0.1 to 1 wt %, 0.25 to 10 wt %, 0.25 to 5 wt %, 0.25 to 3 wt %, 0.25 to 2 wt %, 0.25 to 1 wt %, 0.5 to 10 wt %, 0.5 to 5 wt %, 0.5 to 3 wt %, 0.5 to 2 wt %, 0.5 to 1 wt %, 0.75 to 10 wt %, 0.75 to 5 wt %, 0.75 to 3 wt %, 0.75 to 2 wt % or 0.75 to 1 wt %.

The oil-in-water type composition may further include one or more selected from the group consisting of a surfactant, a wax, a butter, a thickener (stabilizer), a higher alcohol, and a higher fatty acid.

The surfactant may include one or more selected from the group consisting of glyceryl stearate SE, cetearyl alcohol, cetearyl glucoside, glyceryl stearate, PEG-100 stearate, glyceryl stearate/PEG-100 stearate, C14-22 alcohol, C12-20 alkyl glucoside, C14-22 alcohol/C12-20 alkyl glucoside, polyglyceryl-3 methyl glucose distearate, polyglyceryl-6 stearate, polyglyceryl-6 behenate, polyglyceryl-6 stearate/polyglyceryl-6 behenate, C12-16 alcohol, palmitic acid, hydrogenated lecithin, and C2-16 alcohol/palmitic acid/hydrogenated lecithin, but is not limited thereto.

The surfactant may be included in an amount of 0.01 to 10 wt % based on the total weight of the composition, specifically, may be included in an amount of 0.01 to 10 wt %, and specifically 0.01 to 10 wt %, 0.01 to 5 wt %, 0.01 to 4 wt %, 0.01 to 3 wt %, 0.01 to 2 wt %, 0.01 to 1.5 wt %, 0.01 to 1 wt %, 0.1 to 10 wt %, 0.1 to 5 wt %, 0.1 to 4 wt %, 0.1 to 3 wt %, 0.1 to 2 wt %, 0.1 to 1.5 wt %, 0.1 to 1 wt %, 0.5 to 10 wt %, 0.5 to 5 wt %, 0.5 to 4 wt %, 0.5 to 3 wt %, 0.5 to 2 wt %, 0.5 to 1.5 wt %, 0.5 to 1 wt %, 1 to 10 wt %, 1 to 5 wt %, 1 to 4 wt %, 1 to 3 wt %, 1 to 2 wt %, 1 to 1.5 wt %, 1.5 to 10 wt %, 1.5 to 5 wt %, 1.5 to 4 wt %, 1.5 to 3 wt %, 1.5 to 2 wt %, 2 to 10 wt %, 2 to 5 wt %, 2 to 4 wt %, 2 to 3 wt %, 2.5 to 10 wt %, 2.5 to 5 wt %, 2.5 to 4 wt %, 2.5 to 3 wt %, 3 to 10 wt %, 3 to 5 wt % or 3 to 4 wt %.

The surfactant may not include one or more selected from the group consisting of polysorbate 60 and sorbitan sesquioleate.

The thickener may include one or more selected from the group consisting of guar gum, carrageenan, xanthan gum, carboxymethylcellulose, hydroxyethylcellulose, hydroxypropylcellulose, polyacrylate-13, polyacrylate crosspolymer-6, polyacrylamide, sodium acrylate/sodium acryloyldimethyl taurate copolymer, and hydroxyethyl acrylate/sodium acryloyldimethyl taurate copolymer for the purpose of controlling the viscosity of the continuous phase.

The thickener may be included in an amount of 0.01 to 10 wt % based on the total weight of the composition, and specifically, may be included in an amount of 0.01 to 10 wt %, 0.01 to 5 wt %, 0.01 to 4 wt %, 0.01 to 3 wt %, 0.01 to 2 wt %, 0.01 to 1.5 wt %, 0.01 to 1 wt %, 0.1 to 10 wt %, 0.1 to 5 wt %, 0.1 to 4 wt %, 0.1 to 3 wt %, 0.1 to 2 wt %, 0.1 to 1.5 wt %, 0.1 to 1 wt %, 0.5 to 10 wt %, 0.5 to 5 wt %, 0.5 to 4 wt %, 0.5 to 3 wt %, 0.5 to 2 wt %, 0.5 to 1.5 wt %, 0.5 to 1 wt %, 0.7 to 10 wt %, 0.7 to 5 wt %, 0.7 to 4 wt %, 0.7 to 3 wt %, 0.7 to 2 wt %, 0.7 to 1.5 wt %, 0.7 to 1 wt %, 0.9 to 10 wt %, 0.9 to 5 wt %, 0.9 to 4 wt %, 0.9 to 3 wt %, 0.9 to 2 wt %, 0.9 to 1.5 wt %, 0.9 to 1 wt %.

In an embodiment, the cosmetic composition may not include carbomer as a thickener.

The wax may include one or more selected from the group consisting of beeswax, candelilla wax, carnauba wax, multiwax, paraffin wax, microcrystal wax, and ceresin wax.

The butter may include one or more selected from the group consisting of shea butter, mango seed butter, cacao seed butter, cupuacu seed butter, palm seed butter, trichilia emetica seed butter, kokum seed butter, borneo tallow seed butter and sal tree seed butter.

The higher alcohol may include one or more selected from the group consisting of cetyl alcohol, stearyl alcohol, cetearyl alcohol, behenyl alcohol, and batyl alcohol.

The higher fatty acid may include one or more selected from the group consisting of stearic acid, isostearic acid, myristic acid, and palmitic acid.

The cosmetic composition may further include purified water (solvent), a moisturizer, a pH adjuster, a neutralizer, a chelating agent, and/or a preservative, etc. in addition to the components disclosed above.

The preservative may include, one or more selected from the group consisting of phenoxyethanol and hexanediol, but is not limited thereto.

The moisturizer may include, glycerin, but is not limited thereto.

The cosmetic composition may further include oil and/or silicone-based elastomer in addition to the components disclosed above.

The oil is not limited to any type as long as it is an oil commonly used in cosmetics. For example, the oil may be one or more selected from the group consisting of ester-based oil, hydrocarbon-based oil, silicone-based oil, vegetable oil, and triglyceride.

The ester-based oil may be an ester compound of a fatty acid and a fatty alcohol. For example, the ester-based oil may include isopropyl myristate, isopropyl palmitate, ethylhexyl palmitate, cetyl octanoate, cetyl 2-ethyl hexanoate, 2-octyl dodecyl myristate, di-isostearyl malate, etc.

The hydrocarbon-based oil may include petroleum oils such as liquid paraffin, petrolatum, isoparaffin, etc.; synthetic oils such as hydrogenated polydecene, synthetic squalane, polybutene, etc.; and plant oils such as vegetable squalane hydrogenated squalane, etc. and the like.

The silicone-based oil may be an oil having a siloxane bond (Si—O—Si). For example, the silicone-based oil may include dimethicone, dimethiconol, phenyl trimethicone, disiloxane, cyclomethicone, cyclopentasiloxane, cyclohexasiloxane, etc.

The vegetable oil may be an oil obtained from a plant that exists in nature. For example, the vegetable oil may include olive oil, meadowfoam seed oil, rosehip fruit oil, avocado oil, camellia oil, apple seed oil, grape seed oil, sunflower seed oil, macadamia nut oil, coconut oil, castor oil, jojoba seed oil, caprylic/capric triglyceride, etc.

In an embodiment, the cosmetic composition may be an oil-in-water type cosmetic composition including 1) an aqueous phase portion including a cell culture medium, albumin and/or a thickener; and 2) an oil phase portion including a surfactant.

The aqueous phase portion of the cosmetic composition may further include one or more selected from the group consisting of purified water, a moisturizer, and a preservative.

The oil phase portion of the cosmetic composition may further include one or more selected from the group consisting of oil, higher alcohol, higher fatty acid, butter, and wax.

In an embodiment, the cosmetic composition may be an O/W (oil-in-water) emulsion formulation. The cosmetic composition of the present disclosure may be formulated as, for example, a liquid product, lotion, essence, cream, sun lotion, sunscreen, makeup base, foundation, BB cream, stick product, or balm type product, but is not limited thereto. Additionally, depending on the formulation to be prepared, oil, water, surfactants, moisturizers, low grade alcohols, thickeners, chelating agents, preservatives, fragrances, etc. may be selected and added to the composition. Additionally, the composition may also include sunscreen, light scattering agent, etc., and its formulation and additives are not limited to the above descriptions. Additionally, the components may be introduced in amounts generally used in the field of dermatology.

The cosmetic composition may include additional components commonly used in cosmetics, for example, typical auxiliaries such as stabilizers, solubilizers, thickeners, dispersants, fragrances, fillers, preservatives, neutralizers, sweeteners, vitamins, antioxidants, and chelating agents. A person of ordinary skill in the art may select any additional component and/or its amount such that the advantageous properties of the composition according to the present specification are not adversely affected or are substantially unaffected by the anticipated addition.

The cosmetic composition may be a water-in-oil (W/O) type.

The cell culture medium and albumin may be included in an amount of 0.0001 to 10 wt % based on the total weight of the composition, and specifically, may be included in an amount of 0.0001 to 10 wt %, 0.0001 to 5 wt %, 0.0001 to 3 wt %, 0.0001 to 2 wt %, 0.0001 to 1 wt %, 0.001 to 10 wt %, 0.001 to 5 wt %, 0.001 to 3 wt %, 0.001 to 2 wt %, 0.001 to 1 wt %, 0.01 to 10 wt %, 0.01 to 5 wt %, 0.01 to 3 wt %, 0.01 to 2 wt %, 0.01 to 1 wt %, 0.1 to 10 wt %, 0.1 to 5 wt %, 0.1 to 3 wt %, 0.1 to 2 wt %, 0.1 to 1 wt %, 0.25 to 10 wt %, 0.25 to 5 wt %, 0.25 to 3 wt %, 0.25 to 2 wt %, 0.25 to 1 wt %, 0.5 to 10 wt %, 0.5 to 5 wt %, 0.5 to 3 wt %, 0.5 to 2 wt %, 0.5 to 1 wt %, 0.75 to 10 wt %, 0.75 to 5 wt %, 0.75 to 3 wt %, 0.75 to 2 wt % or 0.75 to 1 wt %.

The water-in-oil type composition may further include one or more selected from the group consisting of a surfactant, a wax, a butter, a thickener (stabilizer), a higher alcohol, and a higher fatty acid.

In an embodiment, the water-in-oil type composition may include a cell culture medium, albumin, a surfactant, a wax, and a thickener (stabilizer).

The surfactant may include one or more selected from the group consisting of PEG-8 dimethicone, PEG-9 polydimethylsiloxyethyl dimethicone, PEG-10 dimethicone, PEG-12 dimethicone, PEG-11 methyl ether dimethicone, cetyl PEG/PPG dimethicone, cetyl PEG/PPG-10/1 dimethicone, bis-PEG/PPG-14/14 dimethicone, bis-PEG/PPG-20/20 dimethicone, PEG/PPG-18/18 dimethicone, PEG/PPG-19/19 dimethicone, PEG/PPG-20/20 dimethicone, polyglyceryl-3 polydimethylsiloxyethyl dimethicone, lauryl polyglyceryl-3 polydimethylsiloxyethyl dimethicone, and lauryl PEG-9 polydimethylsiloxyethyl dimethicone, but is not limited thereto.

The surfactant may be included in an amount of 0.01 to 10 wt % based on the total weight of the composition, and specifically, may be included in an amount of 0.01 to 10 wt %, 0.01 to 5 wt %, 0.01 to 4 wt %, 0.01 to 3 wt %, 0.01 to 2 wt %, 0.01 to 1 wt %, 0.1 to 10 wt %, 0.1 to 5 wt %, 0.1 to 4 wt %, 0.1 to 3 wt %, 0.1 to 2 wt %, 0.1 to 1 wt %, 0.5 to 10 wt %, 0.5 to 5 wt %, 0.5 to 4 wt %, 0.5 to 3 wt %, 0.5 to 2 wt %, 0.5 to 1 wt %, 1 to 10 wt %, 1 to 5 wt %, 1 to 4 wt %, 1 to 3 wt %, 1 to 2 wt %, 2 to 10 wt %, 2 to 5 wt %, 2 to 4 wt %, 2 to 3 wt %, 3 to 10 wt %, 3 to 5 wt % or 3 to 4 wt %.

The wax may include one or more selected from the group consisting of beeswax, candelilla wax, carnauba wax, multiwax, paraffin wax, microcrystal wax, and ceresin wax, but is not limited thereto.

The wax may be included in an amount of 0.01 to 10 wt % based on the total weight of the composition, and specifically, may be included in an amount of 0.01 to 10 wt %, 0.01 to 5 wt %, 0.01 to 4 wt %, 0.01 to 3 wt %, 0.01 to 2 wt %, 0.01 to 1.5 wt %, 0.01 to 1 wt %, 0.1 to 10 wt %, 0.1 to 5 wt %, 0.1 to 4 wt %, 0.1 to 3 wt %, 0.1 to 2 wt %, 0.1 to 1.5 wt %, 0.1 to 1 wt %, 0.5 to 10 wt %, 0.5 to 5 wt %, 0.5 to 4 wt %, 0.5 to 3 wt %, 0.5 to 2 wt %, 0.5 to 1.5 wt %, 0.5 to 1 wt %, 1 to 10 wt %, 1 to 5 wt %, 1 to 4 wt %, 1 to 3 wt %, 1 to 2 wt % or 1 to 1.5 wt %.

The thickener may include one or more selected from the group consisting of hectorite, quaternium-18 hectorite, stearalkonium hectorite, and disteardimonium hectorite, for the purpose of a viscosity control agent and stabilizer of the continuous phase, but is not limited thereto.

The thickener (stabilizer) may be included in an amount of 0.01 to 10 wt % based on the total weight of the composition, and specifically, may be included in an amount of 0.01 to 10 wt %, 0.01 to 5 wt %, 0.01 to 4 wt %, 0.01 to 3 wt %, 0.01 to 2 wt %, 0.01 to 1.5 wt %, 0.01 to 1 wt %, 0.1 to 10 wt %, 0.1 to 5 wt %, 0.1 to 4 wt %, 0.1 to 3 wt %, 0.1 to 2 wt %, 0.1 to 1.5 wt %, 0.1 to 1 wt %, 0.5 to 10 wt %, 0.5 to 5 wt %, 0.5 to 4 wt %, 0.5 to 3 wt %, 0.5 to 2 wt %, 0.5 to 1.5 wt %, 0.5 to 1 wt %, 1 to 10 wt %, 1 to 5 wt %, 1 to 4 wt %, 1 to 3 wt %, 1 to 2 wt % or 1 to 1.5 wt %.

The butter may include one or more selected from the group consisting of shea butter, mango seed butter, cacao seed butter, cupuacu seed butter, palm seed butter, trichilia emetica seed butter, kokum seed butter, borneo tallow seed butter and sal tree seed butter, but is not limited thereto.

The higher alcohol may include one or more selected from the group consisting of cetyl alcohol, stearyl alcohol, behenyl alcohol, and batyl alcohol, but is not limited thereto.

The higher fatty acid may include one or more selected from the group consisting of stearic acid, isostearic acid, myristic acid and palmitic acid, but is not limited thereto.

The cosmetic composition may further include oil and/or silicone-based elastomer in addition to the components disclosed above.

The oil is not limited to any type as long as it is an oil commonly used in cosmetics. For example, the oil may be one or more selected from the group consisting of ester-based oil, hydrocarbon-based oil, silicone-based oil, vegetable oil, and triglyceride.

The ester-based oil may be an ester compound of a fatty acid and a fatty alcohol. For example, the ester-based oil may include isopropyl myristate, isopropyl palmitate, ethylhexyl palmitate, cetyl octanoate, cetyl 2-ethyl hexanoate, 2-octyl dodecyl myristate, di-isostearyl malate, etc.

The hydrocarbon-based oil may include petroleum oils such as liquid paraffin, petrolatum, isoparaffin, etc.; synthetic oils such as hydrogenated polydecene, synthetic squalane, polybutene, etc.; and plant oils such as vegetable squalane hydrogenated squalane, etc. and the like.

The silicone-based oil may be an oil having a siloxane bond (Si—O—Si). For example, the silicone-based oil may include dimethicone, dimethiconol, phenyl trimethicone, disiloxane, cyclomethicone, cyclopentasiloxane, cyclohexasiloxane, etc.

The vegetable oil may be an oil obtained from a plant that exists in nature. For example, the vegetable oil may include olive oil, meadowfoam seed oil, rosehip fruit oil, avocado oil, camellia oil, apple seed oil, grape seed oil, sunflower seed oil, macadamia nut oil, coconut oil, castor oil, jojoba seed oil, caprylic/capric triglyceride, etc.

The silicone-based elastomer may refer to a substance in which a silicone cross-linked product is swelled in silicone oil and various oils. The silicone-based elastomer may include a silicone-based crosspolymer. The silicone-based crosspolymer may include, dimethicone crosspolymer, vinyl dimethicone crosspolymer, etc., but is not limited thereto. The silicone-based elastomer may include a dimethicone/vinyl dimethicone crosspolymer.

The cosmetic composition may further include purified water, a moisturizer, a pH adjuster, a chelating agent, and/or a preservative, etc. in addition to the components disclosed above.

The preservative may include, one or more selected from the group consisting of propanediol, ethylhexylglycerin, 1,2-hexanediol, and pentylene glycol, but is not limited thereto.

The moisturizer may include, glycerin, but is not limited thereto.

The chelating agent may include disodium EDTA, but is not limited thereto.

In an embodiment, the cosmetic composition may be a water-in-oil type cosmetic composition including 1) an aqueous phase portion including a cell culture medium and albumin; and 2) an oil phase portion including one or more selected from the group consisting of a thickener (stabilizer), a surfactant, and a wax.

The aqueous phase portion of the cosmetic composition may further include one or more selected from the group consisting of purified water, a chelating agent, a moisturizer, and a preservative.

The oil phase portion of the cosmetic composition may further include one or more selected from the group consisting of oil and silicone-based elastomer.

In an embodiment, the cosmetic composition may be a W/O (water-in-oil) emulsion formulation. The cosmetic composition of the present disclosure may be formulated as, for example, a liquid product, lotion, essence, cream, sun lotion, sunscreen, makeup base, foundation, BB cream, stick product, or balm type product, but is not limited thereto. Additionally, depending on the formulation to be prepared, oil, water, surfactants, moisturizers, low grade alcohols, thickeners, chelating agents, preservatives, fragrances, etc. may be selected and added to the composition. Additionally, the composition may also include sunscreen, light scattering agent, etc., and its formulation and additives are not limited to the above descriptions. Additionally, the components may be introduced in amounts generally used in the field of dermatology.

The cosmetic composition may include additional components commonly used in cosmetics, for example, typical auxiliaries such as stabilizers, solubilizers, thickeners, dispersants, fragrances, fillers, preservatives, neutralizers, sweeteners, vitamins, antioxidants, and chelating agents. A person of ordinary skill in the art may select any additional component and/or its amount such that the advantageous properties of the composition according to the present specification are not adversely affected or are substantially unaffected by the anticipated addition.

Another aspect is to provide a topical skin composition including a cell culture medium and albumin as active components. The same parts of the description above applies equally to the composition.

The topical skin composition may be for improving skin condition.

The improvement in skin condition may include one or more selected from the group consisting of skin moisturization, skin aging improvement, skin barrier strengthening, skin wrinkle improvement, skin elasticity enhancement, skin texture improvement, skin transparency enhancement, skin discoloration and blemish removal, skin whitening, and skin regeneration.

The topical skin composition may be a cream, gel, ointment, skin emulsifier, skin suspension, transdermal delivery patch, drug-containing bandage, lotion, or a combination thereof. The topical skin composition according to each formulation may be appropriately selected and mixed by a person of ordinary skill in the art according to the formulation or intended use of other topical skin preparations, etc., in addition to the composition of the present disclosure, without difficulty, and in this case, a synergistic effect may occur when applied simultaneously with other raw materials.

The topical skin preparation may be appropriately formulated with components commonly used in topical preparations such as cosmetics or pharmaceuticals, etc., for example, aqueous components, oil-based components, powder components, alcohols, moisturizers, thickeners, ultraviolet absorbers, whitening agents, preservatives, antioxidants, surfactants, fragrances, colorants, various skin nutrients, or a combination thereof, according to need.

The topical preparation may also be appropriately formulated with sodium edetate, trisodium edetate, sodium citrate, sodium polyphosphate, sodium metaphosphate, metal sequestrants such as gluconic acid, etc., caffeine, tannins, bellapamil, licorice extract, glabridin, hot water extract of karin fruit, and various herbal medicines, acetic acid tocopherol, glycolytic acid, tranexamic acid and derivatives thereof or salts thereof, etc., vitamin C, magnesium ascorbate phosphate, ascorbic acid glucoside, arbutin, kojic acid, glucose, fructose, trehalose, etc., and other sugars, etc.

The topical skin composition may be an anti-inflammatory composition.

The topical skin composition may be an oil-in-water type or a water-in-oil type.

Another aspect is to provide a method of improving the solubility of a composition including a cell culture medium and albumin, including: 1) a step of dissolving components of a cell culture medium and albumin in a solvent; and 2) a step of adding a solubility enhancer to the solution obtained in the above step and mixing the same. The same aspects as described above apply equally to the method.

The method may include a step of adjusting the pH of the solution to a range of 3.0 to 9.0 by adding the solubility enhancer, wherein the solubility enhancer may be a pH adjuster.

In an embodiment, the composition may be adjusted to have a pH of 3.0 to 9.0, and more specifically adjusted to have a pH in the range of 6.0 to 8.0, and even more specifically adjusted to have a pH in the range of 3.0 to 9.0, 3.0 to 8.5, 3.0 to 8.0, 3.0 to 7.8, 3.0 to 7.6, 3.0 to 7.5, 3.5 to 9.0, 3.5 to 8.5, 3.5 to 8.0, 3.5 to 7.8, 3.5 to 7.6, 3.5 to 7.5, 4.0 to 9.0, 4.0 to 8.5, 4.0 to 8.0, 4.0 to 7.8, 4.0 to 7.6, 4.0 to 7.5, 4.5 to 9.0, 4.5 to 8.5, 4.5 to 8.0, 4.5 to 7.8, 4.5 to 7.6, 4.5 to 7.5, 5.0 to 9.0, 5.0 to 8.5, 5.0 to 8.0, 5.0 to 7.8, 5.0 to 7.6, 5.0 to 7.5, 5.5 to 9.0, 5.5 to 8.5, 5.5 to 8.0, 5.5 to 7.8, 5.5 to 7.6, 5.5 to 7.5, 6.0 to 9.0, 6.0 to 8.5, 6.0 to 8.0, 6.0 to 7.8, 6.0 to 7.6, 6.0 to 7.5, 6.5 to 9.0, 6.5 to 8.5, 6.5 to 8.0, 6.5 to 7.8, 6.5 to 7.6, 6.5 to 7.5, 7.0 to 9.0, 7.0 to 8.5, 7.0 to 8.0, 7.0 to 7.8, 7.0 to 7.6, 7.0 to 7.5, 7.2 to 9.0, 7.2 to 8.5, 7.2 to 8.0, 7.2 to 7.8, 7.2 to 7.6, 7.2 to 7.5, 7.4 to 9.0, 7.4 to 8.5, 7.4 to 8.0, 7.4 to 7.8, 7.4 to 7.6, 7.4 to 7.5, 7.5 to 9.0, 7.5 to 8.5, 7.5 to 8.0, 7.5 to 7.8, or 7.5 to 7.6.

Another aspect is to provide a composite powder including a cell culture medium, albumin and a porous polymer.

In the composite powder, the cell culture medium and albumin may be impregnated into the porous polymer, and specifically, may be evenly impregnated into the surface and inside of the spherical porous polymer.

The porous polymer may include one or more selected from the group consisting of polymethyl methacrylate [PMMA: poly-(methyl methacrylate)], polyvinyl pyrrolidone [PVP: poly-(vinyl pyrrolidone)], polycaprolactone [PCL: poly-(caprolactone)], polymethylsilsesquioxane [PMSQ: polymethylsilsesquioxane], silica, and poly-L-lactic acid [PLLA: poly-(L-Lactic Acid)], and specifically, may include polymethyl methacrylate. Additionally, the porous polymer may be a porous polymer powder.

The composite powder may have an enhanced level of homogenization, and specifically, may have an enhanced level of homogenization compared to a composition including a cell culture medium and albumin but not including a porous polymer.

Specifically, the homogenization level of the composite powder may be enhanced by 10% or more, 30% or more, 50% or more, 70% or more, 100% or more, 150% or more, 200% or more, or 300% or more, compared to a composition including a cell culture medium and albumin and not including a porous polymer, and specifically, may be enhanced by 10% to 300%, 10% to 200%, 10% to 150%, 10% to 100%, 10% to 70%, 10% to 50%, 30% to 300%, 30% to 200%, 30% to 150%, 30% to 100%, 30% to 70%, 30% to 50%, 50% to 300%, 50% to 200%, 50% to 150%, 50% to 100%, or 50% to 70%.

Another aspect is to provide a method of preparing a composite powder including a cell culture medium and albumin, including: 1) a step of dissolving or dispersing a cell culture medium, albumin, and a porous polymer in a solvent; and 2) a step of spray-drying the solution or dispersion. The same aspects as described above apply equally to the method.

In the above method, ‘step 1)’ is a step preparing a solution in which the cell culture medium, albumin, and porous polymer are dissolved, mixed, or dispersed, and may specifically include a step of dissolving a cell culture medium and albumin in a solvent and mixing or dispersing a porous polymer in the solution.

The solvent may be an organic solvent, and may include one or more selected from the group consisting of ethanol, methanol, acetone and dichloromethane, and specifically may include methanol and dichloromethane.

The solvent may include methanol and dichloromethane in a volume ratio of 0.5:9.5 to 3:7, and specifically, may include in a volume ratio of 0.5:9.5 to 3:7, 0.5:9.5 to 2.5:7.5, 0.5:9.5 to 2:8, 0.5:9.5 to 1.5:8.5, 1.0:9.0 to 3:7, 1.0:9.0 to 2.5:7.5, 1.0:9.0 to 2:8, 1.0:9.0 to 1.5:8.5, 1.5:8.5 to 3:7, 1.5:8.5 to 2.5:7.5 or 1.5:8.5 to 2:8.

In the above method, ‘step 2)’ may include a step of preparing a composite powder in which the cell culture medium and albumin are evenly impregnated into the porous polymer powder by spraying the mixture or dispersion of ‘step 1)’ by spray-drying.

The term “spray-drying” in the present specification refers to a drying method in which a mixture of solid and liquid is exposed to a hot air stream to instantaneously evaporate the liquid in the solution phase to obtain solid fine particles. For the spray-drying step, a process to generate a high temperature hot air stream to evaporate the liquid and a process to atomize and spray the liquid and solid mixture using a nozzle or spray are required.

The spray-drying step may be conducted by one or more selected from the group consisting of a disk type spray-dryer and a nozzle type spray dryer.

In the spray-drying step, the air inlet temperature of the spray-dryer may be 80 to 120° C., and specifically, may be 80 to 120° C., 80 to 115° C., 80 to 110° C., 80 to 105° C., 80 to 100° C., 80 to 95° C., 85 to 120° C., 85 to 115° C., 85 to 110° C., 85 to 105° C., 85 to 100° C., 85 to 95° C., 90 to 120° C., 90 to 115° C., 90 to 110° C., 90 to 105° C., 90 to 100° C., 90 to 95° C., 95 to 120° C., 95 to 115° C., 95 to 110° C., 95 to 105° C. or 95 to 100° C.

In the spray-drying step, the chamber temperature of the spray-dryer may be 60 to 90° C., and specifically, may be 60 to 90° C., 60 to 85° C., 60 to 80° C., 60 to 75° C., 65 to 90° C., 65 to 85° C., 65 to 80° C., 65 to 75° C., 70 to 90° C., 70 to 85° C., 70 to 80° C. or 70 to 75° C.

In the spray-drying step, the disk rotation speed of the disk type spray-dryer may be 6,000 to 10,000 rpm/min, and specifically, may be 6,000 to 10,000 rpm/min, 6,000 to 9,500 rpm/min, 6,000 to 9,000 rpm/min, 6,000 to 8,500 rpm/min, 6,000 to 8,000 rpm/min, 6,000 to 7,500 rpm/min, 6,500 to 10,000 rpm/min, 6,500 to 9,500 rpm/min, 6,500 to 9,000 rpm/min, 6,500 to 8,500 rpm/min, 6,500 to 8,000 rpm/min, 6,500 to 7,500 rpm/min, 7,000 to 10,000 rpm/min, 7,000 to 9,500 rpm/min, 7,000 to 9,000 rpm/min, 7,000 to 8,500 rpm/min, 7,000 to 8,000 rpm/min, 7,000 to 7,500 rpm/min, 7,500 to 10,000 rpm/min, 7,500 to 9,500 rpm/min, 7,500 to 9,000 rpm/min, 7,500 to 8,500 rpm/min or 7,500 to 8,000 rpm/min.

In the spray-drying step, the flow rate may be 10 to 20 kg/h, and specifically, may be 10 to 20 kg/h, 10 to 19 kg/h, 10 to 18 kg/h, 10 to 17 kg/h, 12 to 20 kg/h, 12 to 19 kg/h, 12 to 18 kg/h, 12 to 17 kg/h, 14 to 20 kg/h, 14 to 19 kg/h, 14 to 18 kg/h, 14 to 17 kg/h, 16 to 20 kg/h, 16 to 19 kg/h, 16 to 18 kg/h or 16 to 17 kg/h.

The method may be for enhancing the level of homogenization of a composition including a cell culture medium and albumin, and specifically for homogenizing particles despite differences in crystal form, size and specific gravity of the components.

Another aspect is to provide a composition including a composite powder including a cell culture medium, albumin, and a porous polymer. The same parts of the description above applies equally to the composition.

The composite powder may be prepared using the foregoing method of preparing a composite powder.

The composition may be a cosmetic composition.

The formulation of the cosmetic composition is not particularly limited, and for example, may have any one formulation selected from among toners, lotions, essences, creams, packs, foundations, patches, hair tonics, microneedle patches, and makeup bases, but may not be limited thereto.

The cosmetic composition may add a substance arbitrarily selected according to the formulation or intended use of the cosmetic, but may not be limited thereto. For example, purified water, oil, surfactants, moisturizers, higher alcohols, thickeners, chelating agents, pigments, fatty acids, antioxidants, preservatives, waxes, pH adjusters, fragrances, etc. may be added, but may not be limited thereto.

When the formulation of the cosmetic composition is a paste, cream or gel, it may include, animal oil, vegetable oil, wax, paraffin, starch, tragacanth, cellulose derivatives, polyethylene glycol, silicone, bentonite, silica, talc or zinc oxide, etc. as a carrier component, but may not be limited thereto.

When the formulation of the cosmetic composition is a powder or spray, it may include lactose, talc, silica, aluminum hydroxide, calcium silicate or polyamide powder as a carrier component, and in particular, if it is a spray, may further include a propellant such as chlorofluorohydrocarbon, propane/butane or dimethyl ether, but may not be limited thereto.

When the formulation of the cosmetic composition is a solution or an emulsion, it may include a solvent, a solubilizer or an emulsifier as a carrier component, and may include, for example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan, but may not be limited thereto.

When the formulation of the cosmetic composition is a suspension, it may include, a liquid diluent such as water, ethanol or propylene glycol as a carrier component, a suspending agent such as ethoxylated isostearyl alcohol, polyoxyethylene sorbitol ester and polyoxyethylene sorbitan ester, microcrystalline cellulose, aluminum methahydroxide, bentonite, agar or tragacanth, etc., but may not be limited thereto.

When the formulation of the cosmetic composition is a surfactant-containing cleanser, the carrier component may include, an aliphatic alcohol sulfate, an aliphatic alcohol ether sulfate, a sulfosuccinic acid monoester, an isethionate, an imidazolinium derivative, methyl taurate, a sarcosinate, a fatty acid amide ether sulfate, an alkylamidobetaine, an aliphatic alcohol, a fatty acid glyceride, a fatty acid diethanolamide, a vegetable oil, a lanolin derivative, or an ethoxylated glycerol fatty acid ester, etc., but may not be limited thereto.

As an example of the cosmetic composition formulation, it may be prepared into a formulation of skincare products including softening lotion, astringent lotion, nourishing lotion, lotion, gel, cream, essence, eye essence, eye cream, nourishing cream, massage cream, clay-type pack, and mask pack; makeup products including lipstick, lip tint, lip gloss, lip pencil, eye shadow, foundation, powder, concealer, eyeliner, eye shadow, mascara; cleansing products including eye makeup remover, makeup remover, foam cleanser, cleansing cream, cleansing oil, cleansing water, hand sanitizer, hand wash, hand scrub, body wash, body scrub, shaving lotion, soap, and wet wipe; body care products including body lotion, body oil, body mist, body essence, hand cream, foot cream, and wax-type hair removal agent; baby cosmetics including baby lotion, baby cream, and diaper rash cream; sun care products including sunscreen, sun stick, sun spray, and artificial tanning product; hair products including shampoo, hair conditioner, hair treatment cream, hair styling gel, foam, hair mousse, hair spray, styling lotion, styling cream, hair essence, hair dye, hair bleaching cream, and curl activator gel; nail care products including nail varnish, nail polish, cuticle remover, and cuticle cream; personal care products including toothpaste, mouthwash, oral rinse, oral film, and gum; perfume, plant extract, deodorant, and antiperspirant.

The composition may be a topical skin composition.

The topical skin composition may be a cream, gel, ointment, skin emulsifier, skin suspension, transdermal delivery patch, drug-containing bandage, lotion, or a combination thereof. The topical skin composition according to each formulation may be appropriately selected and mixed by a person of ordinary skill in the art according to the formulation or intended use of other topical skin preparations, etc., in addition to the composition of the present disclosure, without difficulty, and in this case, a synergistic effect may occur when applied simultaneously with other raw materials.

The topical skin preparation may be appropriately formulated with components commonly used in topical preparations such as cosmetics or pharmaceuticals, etc., for example, aqueous components, oil-based components, powder components, alcohols, moisturizers, thickeners, ultraviolet absorbers, whitening agents, preservatives, antioxidants, surfactants, fragrances, colorants, various skin nutrients, or a combination thereof, according to need.

The topical preparation may also be appropriately formulated with sodium edetate, trisodium edetate, sodium citrate, sodium polyphosphate, sodium metaphosphate, metal sequestrants such as gluconic acid, etc., caffeine, tannins, bellapamil, licorice extract, glabridin, hot water extract of karin fruit, and various herbal medicines, acetic acid tocopherol, glycolytic acid, tranexamic acid and derivatives thereof or salts thereof, etc., vitamin C, magnesium ascorbate phosphate, ascorbic acid glucoside, arbutin, kojic acid, glucose, fructose, trehalose, etc., and other sugars, etc.

### Advantageous Effects of Invention

The cosmetic composition according to the present disclosure has been confirmed to effectively improve skin conditions such as skin moisturization, skin aging improvement, skin barrier strengthening, skin wrinkle improvement, skin elasticity enhancement, skin texture improvement, skin transparency enhancement, skin discoloration and blemish removal, skin whitening, and skin regeneration, therefore, may be usefully utilized as a cosmetic composition for improving skin conditions and may be used as a new cosmetic.

In addition, the cosmetic composition according to the present disclosure has been confirmed to effectively improve skin conditions by inhibiting the expression of inflammatory cytokines IL-6 and IL-8, etc., thereby alleviating inflammation, and therefore, may be usefully utilized as a cosmetic composition for improving skin conditions and may be used as a new cosmetic.

In addition, in the case of the composition of the present disclosure, it has been confirmed that the cell culture medium components and albumin may be effectively and completely dissolved without residue by adding a solubility enhancer. Therefore, through the improvement in solubility, the composition may be applied to various product groups such as toner, lotion, essence, cream, and pack, etc.

In addition, the present disclosure relates to an oil-in-water type cosmetic composition for skin improvement containing a cell culture medium and albumin, the cosmetic composition of the present disclosure, while including the cell culture medium and albumin, provides stability of the formulation, so that it may be usefully used as an oil-in-water type cosmetic composition for skin improvement that may be smoothly spread and provides a feeling of nutrition without being sticky after absorption.

In addition, the present disclosure relates to an water-in-oil type cosmetic composition for skin improvement containing a cell culture medium and albumin, the cosmetic composition of the present disclosure, while including the cell culture medium and albumin, provides stability of the formulation, so that it may be usefully used as an cosmetic composition for skin improvement that may be smoothly spread and provides a feeling of nutrition without being sticky after absorption.

In addition, the present disclosure introduces a spray-drying step to prepare a powder in which a cell culture medium and albumin are impregnated into a porous polymer. Accordingly, there is an advantage in that the surface and interior of a spherical porous polymer may be evenly impregnated in a single process while excluding the use of an emulsifier that irritates the skin. In addition, by impregnating an effective component with non-homogeneous specific gravity and crystal form into the inside and outside of a spherical porous polymer, there is an effect wherein the homogeneity is improved, thereby resolving the variation in the content and component of raw materials.

### BRIEF DESCRIPTION OF DRAWINGS

FIG. 1 is a drawing illustrating the results of confirming the inhibitory efficacy of SPM on IL-6, an immune/inflammatory factor.

FIG. 2 is a drawing illustrating the results of confirming the inhibitory efficacy of SPM on IL-8, an immune/inflammatory factor.

FIG. 3 is a drawing illustrating a scanning electron microscope image of a composition including a powder-state cell culture medium and albumin.

FIG. 4 is a drawing illustrating a scanning electron microscope image of PMMA, a porous polymer powder.

FIG. 5 is a drawing illustrating a scanning electron microscope image of PMMA impregnated with cell culture medium and albumin components.

### MODE FOR THE INVENTION

The present disclosure will be explained in more detail in the following examples. However, these examples are for illustrative purposes only and the scope of the present disclosure is not limited to these examples.

### Preparation Example 1: Preparation of Composition Including Cell Culture Medium and Albumin

The components of the cell culture medium and albumin disclosed in Table 1 below were sufficiently dissolved in a solvent (for example, purified water, etc.), and after confirming that all components were dissolved, they were filtered through a 0.2 μm filter to prepare a mixed composition including the following 23 components. Each component disclosed in Table 1 below were all purchased from Sigma-Aldrich (USA), and the combination of 23 components disclosed in Table 1 below is hereinafter referred to as ‘SPM.’

Meanwhile, the concentration disclosed in Table 1 refers to the final concentrations in the solution when the above components are completely dissolved in the solvent.

TABLE 1

| | Concen- |
|---|---|
| | tration |
| Component | (g/L) |
| Glycine | 0.01875 |
| L-alanine | 0.00445 |
| L-arginine hydrochloride | 0.1475 |
| L-cysteine hydrochloride-monohydrate-H₂O | 0.01756 |
| L-glutamine | 0.365 |
| L-histidine hydrochloride-monohydrate | 0.03148 |
| L-lysine hydrochloride | 0.09125 |
| L-methionine | 0.01724 |
| L-proline | 0.01725 |
| L-serine | 0.02625 |
| L-threonine | 0.05345 |
| L-valine | 0.05285 |
| i-inositol | 0.0126 |
| Thiamine hydrochloride | 0.00217 |
| Niacinamide | 0.00202 |
| Pyridoxine hydrochloride | 0.002 |
| Sodium chloride (NaCl) | 3.49775 |
| Sodium bicarbonate (NaHCO₃) | 2.438 |
| Potassium chloride (KCl) | 0.3118 |
| Calcium chloride (CaCl₂) (anhydrous) | 0.1166 |
| Sodium phosphate monobasic monohydrate (NaH₂PO₄—H₂O) | 0.0625 |
| Sodium pyruvate | 0.055 |
| Albumin | 0.1 |

### Experimental Example 1: Confirmation of Skin Condition Improvement Effect

### 1-1. Preparation of Cosmetic Composition Including Cell Culture Medium and Albumin

After weighing each raw material in a beaker according to the stone components and content (wt %) disclosed in Table 2 below. The mixture was uniformly mixed at room temperature using a disperser at 1000 rpm for 30 minutes.

TABLE 2

| | | Comparative |
|---|---|---|
| | Example 1 | Example 1 |
| Purified water | 64.00 | 64.76 |
| SPM | 0.76 | — |
| Citric acid | 0.04 | 0.04 |
| Sodium citrate | 0.01 | 0.01 |
| Sodium hyaluronate | 0.10 | 0.10 |
| Betaine | 1.00 | 1.00 |
| Niacinamide | 2.00 | 2.00 |
| Adenosine | 0.04 | 0.04 |
| Propanediol | 5.00 | 5.00 |
| Glycerin | 15.00 | 15.00 |
| Butylene glycol | 10.00 | 10.00 |
| 1,2-Hexanediol | 2.00 | 2.00 |
| Ethylhexylglycerin | 0.05 | 0.05 |

### 1-2. Evaluation of Skin Texture Improvement Efficacy

To evaluate the skin texture improvement efficacy of the composition including SPM prepared in the Experimental Example 1-1, the following experiment was conducted.

Specifically, a panel of 20 women aged 20 to 59 years were asked to use the test product on their faces for 4 weeks (twice a day). Before instrumental measurement, the subjects waited for 30 minutes under conditions of constant temperature and humidity (20-24° C., 45-55% RH) after washing their faces, and instrumental measurements were conducted prior to product use, as well as 2 weeks and 4 weeks after use.

Skin texture was measured once on the test area/control area (cheek) using Antera 3D CS (Miravex, Ireland), and the average roughness (Ra) was used as evaluation data. The measuring instrument is an equipment capable of capturing the surface image of the skin using a light-emitting diode (LED light source), and may extract data from the three-dimensional shape image of the built-in program, quantify the skin condition, and utilize the image. A decrease in the skin roughness measurement value (Ra) indicates an improvement in skin texture.

As a result of measuring skin texture after 4 weeks of use of Example 1 including the SPM and Comparative Example 1 not including SPM as a control group, it was confirmed that the product containing SPM as an active component had an excellent effect in improving skin texture. (Table 3).

TABLE 3

| Skin Texture Measurement Result | | Test Area | Control Area |
|---|---|---|---|
| Skin Texture | Before Use (A) | 6.530 ± 1.812 | 6.528 ± 1.807 |
| Measurement | After 2 Weeks of Use (B) | 6.142 ± 1.724 | 6.341 ± 1.768 |
| Value (Ra) | After 4 Weeks of Use (C) | 5.788 ± 1.630 | 6.094 ± 1.691 |

| | | |
|---|---|---|
| Change After 2 Weeks of Use (B − A) | −0.388 | −0.187 |
| Change After 4 Weeks of Use (C − A) | −0.742 | −0.434 |

Based on the above results, it was confirmed that skin texture roughness was reduced and improved in the case of a composition containing SPM (a mixture of cell culture medium components and albumin).

### 1-3. Evaluation of Skin Transparency Improvement Efficacy

To evaluate the skin transparency improvement efficacy of the composition including SPM prepared in the Experimental Example 1-1, the following experiment was conducted.

Specifically, a panel of 20 women aged 20 to 59 years were asked to use the test product on their faces for 4 weeks (twice a day). Before instrumental measurement, the subjects waited for 30 minutes under conditions of constant temperature and humidity (20-24° C., 45-55% RH) after washing their faces, and instrumental measurements were conducted prior to product use, as well as 2 weeks and 4 weeks after use.

Skin transparency was measured once at the test area/control area (cheek) using a Translucencymeter (TLS850, Dia-Stron, United Kingdom). The measurement principle is to irradiate a thin beam from an RGB LED Light Source onto the skin and measure the intensity of the scattered light scattered inside the skin using a FOP (Fibre Optic Face Plate). The rate of decrease in the amount of light transmitted through the skin according to distance from the light source increases is represented, and a decrease in the measurement value (Alpah) indicates that skin transparency improves.

As a result of measuring skin transparency after 4 weeks of use of Example 1 including the SPM and Comparative Example 1 not including SPM as a control group, it was confirmed that skin transparency increased in the product containing SPM as an active component. (Table 4).

TABLE 4

| Skin Transparency Measurement Result | | Test Area | Control Area |
|---|---|---|---|
| Skin | Before Use (A) | 0.0257 ± 0.0057 | 0.0255 ± 0.0057 |
| Transparency | After 2 Weeks of Use (B) | 0.0248 ± 0.0055 | 0.0249 ± 0.0055 |
| Measurement | After 4 Weeks of Use (C) | 0.0236 ± 0.0052 | 0.0245 ± 0.0054 |
| Value | | | |
| (alpha) | | | |

| | | |
|---|---|---|
| Change After 2 Weeks of Use (B − A) | −0.0009 | −0.0006 |
| Change After 4 Weeks of Use (C − A) | −0.0021 | −0.0010 |

Based on the above results, it was confirmed that skin transparency was improved in the case of a composition containing SPM (a mixture of cell culture medium components and albumin).

### 1-4. Evaluation of Skin Moisturizing Efficacy

To evaluate the skin moisturization improvement efficacy of the composition including SPM prepared in the Experimental Example 1-1, the following experiment was conducted.

Specifically, a panel of 20 women aged 20 to 59 years were asked to use the test product on their faces for 4 weeks (twice a day). Before instrumental measurement, the subjects waited for 30 minutes under conditions of constant temperature and humidity (20-24° C., 45-55% RH) after washing their faces, and instrumental measurements were conducted prior to product use, as well as 2 weeks and 4 weeks after use.

Skin moisturization was measured using Corneometer CM825 (Courage Khazaka electronic, Germany) on the test area/control area (cheeks), and the average value from three measurements was used as the evaluation data. The measurement principle is to use capacitance, and the difference in dielectric constant between water and the measured substance is presented as a measurement value. An increase in the measurement value (A.U) indicates an improvement in skin moisturization.

As a result of measuring skin moisturization after 4 weeks of use of Example 1 including the SPM and Comparative Example 1 not including SPM as a control group, it was confirmed that the product containing SPM as an active component had an excellent moisturizing effect. (Table 5).

TABLE 5

| Skin Moisture Measurement Result | | Test Area | Control Area |
|---|---|---|---|
| Skin Moisture | Before Use (A) | 34.773 ± 13.178 | 35.283 ± 13.571 |
| Measurement | After 2 Weeks of Use (B) | 41.400 ± 14.561 | 38.507 ± 14.124 |
| Value | After 4 Weeks of Use (C) | 46.426 ± 15.320 | 41.653 ± 14.277 |
| (A.U) | | | |

| | | |
|---|---|---|
| Change After 2 Weeks of Use (B − A) | 6.627 | 3.224 |
| Change After 4 Weeks of Use (C − A) | 11.653 | 6.37 |

Based on the above results, it was confirmed that skin moisturization was significantly enhanced in the case of a composition containing SPM (a mixture of cell culture medium components and albumin).

### 1-5. Evaluation of Skin Discoloration/Blemish Improvement Efficacy

To evaluate the skin discoloration/blemish improvement efficacy of the composition including SPM prepared in the Experimental Example 1-1, the following experiment was conducted.

Specifically, a panel of 20 women aged 20 to 59 years were asked to use the test product on their faces for 4 weeks (twice a day). Before instrumental measurement, the subjects waited for 30 minutes under conditions of constant temperature and humidity (20-24° C., 45-55% RH) after washing their faces, and instrumental measurements were conducted prior to product use, as well as 2 weeks and 4 weeks after use.

Discoloration/blemishes were measured once on the test area/control area (discoloration/blemish area) using Antera 3D CS (Miravex, Ireland), and the affected area (mm²) value in Melanin-Hyperconcentration analysis mode was used as evaluation data. The measurement principle is that it is an equipment that may measure the surface image of the skin using a light-emitting diode (LED light source), and utilizes a built-in program to create a three-dimensional shape image. A decrease in the measurement value (mm²) indicates an improvement in discoloration/blemishes.

As a result of measuring skin discoloration/blemish after 4 weeks of use of Example 1 including the SPM and Comparative Example 1 not including SPM as a control group, it was confirmed that the product containing SPM as an active component had an excellent effect in improving skin discoloration/blemish. (Table 6).

TABLE 6

| Discoloration and Blemish Area | | | |
|---|---|---|---|
| Measurement Result | | Test Area | Control Area |
| Discoloration | Before Use (A) | 36.700 ± 13.998 | 36.700 ± 13.930 |
| and Blemish | After 2 Weeks of Use (B) | 32.250 ± 12.838 | 34.450 ± 13.452 |
| Area | After 4 Weeks of Use (C) | 28.450 ± 12.198 | 31.900 ± 12.998 |
| Measurement | | | |
| Value | | | |
| (mm²) | | | |

| | | |
|---|---|---|
| Change After 2 Weeks of Use (B − A) | −4.45 | −2.25 |
| Change After 4 Weeks of Use (C − A) | −8.25 | −4.8 |

Based on the above results, it was confirmed that the area of discoloration/blemish was reduced and improved in the case of a composition containing SPM (a mixture of cell culture medium components and albumin).

### 1-6. Evaluation of Skin Elasticity Improvement Efficacy

To evaluate the skin elasticity improvement efficacy of the composition including SPM prepared in the Experimental Example 1-1, the following experiment was conducted.

Specifically, a panel of 20 women aged 20 to 59 years were asked to use the test product on their faces for 4 weeks (twice a day). Before instrumental measurement, the subjects waited for 30 minutes under conditions of constant temperature and humidity (20-24° C., 45-55% RH) after washing their faces, and instrumental measurements were conducted prior to product use, as well as 2 weeks and 4 weeks after use.

Skin elasticity was measured using Cutometer Dual MPA580 (Courage Khazaka electronic, Germany), and the test area/control area (cheek) was measured once and used as evaluation data. The measurement principle is to use negative pressure (450 mbar) and measure the length of skin pulled into a 2 mm diameter probe using infrared rays. The R² parameter, which represents the total elasticity of the skin as a total elasticity value, is used as an evaluation indicator, and the closer the R² value, which is a skin elasticity evaluation indicator, gets to 1, the more the skin elasticity improves.

As a result of measuring skin elasticity after 4 weeks of use of Example 1 including the SPM and Comparative Example 1 not including SPM as a control group, it was confirmed that the product containing SPM as an active component had an excellent effect in improving skin elasticity. (Table 7).

TABLE 7

| Skin Elasticity Measurement Result | | Test Area | Control Area |
|---|---|---|---|
| Skin Elasticity | Before Use | 0.649 ± 0.150 | 0.646 ± 0.147 |
| Measurement | | | |
| Value | After 2 Weeks of Use | 0.676 ± 0.156 | 0.660 ± 0.150 |
| (R²) | After 4 Weeks of Use | 0.696 ± 0.159 | 0.671 ± 0.152 |

| | | |
|---|---|---|
| Change After 2 Weeks of Use (B − A) | 0.027 | 0.014 |
| Change After 4 Weeks of Use (C − A) | 0.047 | 0.025 |

Based on the above results, it was confirmed that skin elasticity was increased and improved in the case of a composition containing SPM (a mixture of cell culture medium components and albumin).

### Experimental Example 2: Confirmation of Anti-Inflammatory Factor Inhibition Effect

### 2-1. Confirmation of Anti-Inflammatory Factor Inhibition Effect

To confirm the anti-inflammatory effect of SPM of the above Preparation Example 1, the expression levels of inflammatory cytokines IL-6 and IL-8 were measured in HaCaT keratinocytes.

Specifically, HaCaT Keratinocyte cells were suspended in 2 mL of DMEM medium including 10% FBS, seeded into 6-well plates at 4×10⁵ cells per well, and cultured at 37° C. in 5% CO₂ for 24 hours. Thereafter, the samples were simultaneously added while treating with the stimulating substances Poly IC (10 μg/ml) and IL-4 (10 ng/ml), and incubated at 37° C. for 4 hours to induce overexpression of the inflammatory cytokines IL-6 and IL-8. At this time, 1 μM dexamethasone was used as a positive control substance. Afterwards, the cells were washed with phosphate buffered saline (PBS), mRNA was extracted from within the cells, cDNA was synthesized, and real-time PCR was performed using target templates (primers) to evaluate the expression levels of IL-6 and IL-8.

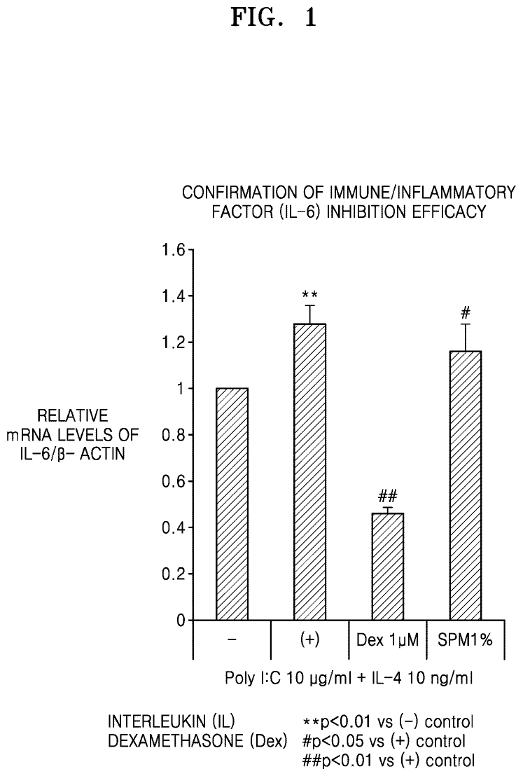

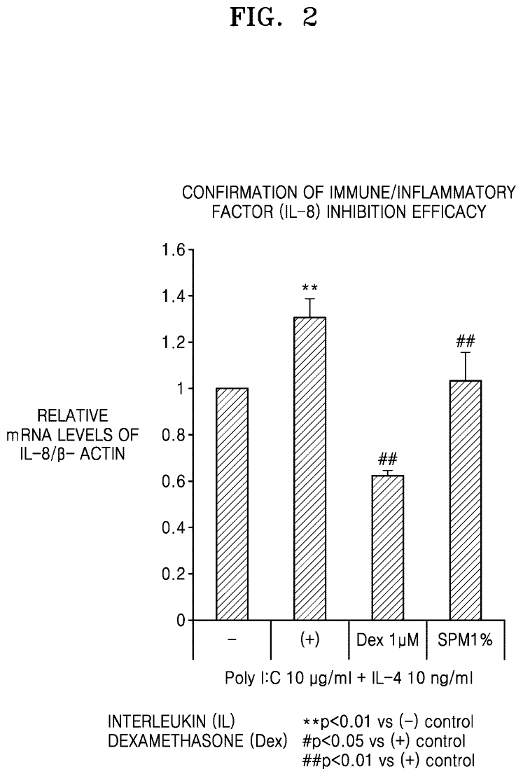

As a result, as shown in FIG. 1 and FIG. 2, the effect of inhibiting the expression of IL-6 and IL-8 was confirmed in the SPM treatment group.

2-2. 2-2.

Preparation of Various Formulations Examples Based on the results of the above experimental examples, a cosmetic composition containing a cell culture medium and albumin according to the present disclosure is presented.

However, the composition of the present disclosure is not intended to be limited to the following formulation examples.

### (1) Formulation Example 1: Softening Lotion (Toner Lotion)

A softening lotion formulation including cell culture medium and albumin was prepared according to a typical method with the components/contents shown in Table 8 below.

TABLE 8

| | Component |
|---|---|
| Content (wt %) | Purified water |
| to 100 | Cell culture |
| | medium/Albumin |
| 0.78 | Butylene glycol |
| 4.00 | Glycerin |
| 4.00 | 1,2-Hexanediol |
| 1.00 | Pentylene glycol |
| 1.00 | Propanediol |
| 5.00 | Polysorbate 20 |
| 0.60 | Ethylhexylglycerin |
| 0.20 | Xanthan Gum |
| Appropriate Amount | Fragrance |
| Appropriate Amount | Citric acid |

### (2) Formulation Example 2: Nourishing Lotion (Milk Lotion)

A nourishing lotion formulation including cell culture medium and albumin was prepared according to a typical method with the components/contents shown in Table 9 below.

TABLE 9

| | Content |
|---|---|
| Component | (wt %) |
| Purified water | to 100 |
| Cell culture medium/Albumin | 0.78 |
| Glycerin | 8.00 |
| Butylene glycol | 3.00 |
| Hydroxyacetophenone | 0.50 |
| Shea butter | 2.00 |
| Avocado oil | 3.00 |
| Coconut oil | 3.00 |
| Coconut butter | 1.00 |
| Cetearyl alcohol | 1.00 |
| Glyceryl stearate | 0.30 |
| Polyglyceryl-3 methyl glucose distearate | 1.20 |
| Cetearyl alcohol/Cetearyl glucoside/Glucose | 0.60 |
| Hydroxyethyl acrylate/Sodium acryloyldimethyl | 1.00 |
| taurate copolymer | |
| Caprylyl glycol | 0.50 |
| Xanthan Gum | Appropriate |
| | Amount |
| Citric acid | Appropriate |
| | Amount |
| Fragrance | Appropriate |
| | Amount |

### (3) Formulation Example 3: Nourishing Essence

A nourishing essence formulation including cell culture medium and albumin was prepared according to a typical method with the components/contents shown in Table 10 below.

TABLE 10

| | Content |
|---|---|
| Component | (wt %) |
| Purified water | to 100 |
| Cell culture medium/Albumin | 0.78 |
| Hydroxyacetophenone | 0.40 |
| Tromethamine | 0.08 |
| Propanediol | 5.30 |
| Glycerin | 3.00 |
| Panthenol | 0.50 |
| 1,2-Hexanediol | 1.20 |
| Methylpropanediol | 4.00 |
| Ammonium acryloyldimethyl taurate/VP Copolymer | 0.30 |
| Carbomer | 0.12 |
| Dipropylene glycol | 3.00 |
| Polyglyceryl-10 Laurate | 0.30 |
| Ethylhexylglycerin | 0.10 |
| Disodium EDTA | Appropriate |
| | Amount |
| Xanthan Gum | Appropriate |
| | Amount |
| Citric acid | Appropriate |
| | Amount |
| Fragrance | Appropriate |
| | Amount |

### (4) Formulation Example 4: Nourishing Cream

A nourishing cream formulation including cell culture medium and albumin was prepared according to a typical method with the components/contents shown in Table 11 below.

TABLE 11

| | Content |
|---|---|
| Component | (wt %) |
| Purified water | to 100 |
| Cell culture medium/Albumin | 0.78 |
| Glycerin | 2.00 |
| Glycerin | 5.00 |
| 1,2-Hexanediol | 1.00 |
| Pentylene glycol | 1.00 |
| Glyceryl stearate | 0.60 |
| Polyglyceryl-3 methyl glucose distearate | 2.00 |
| Cetearyl alcohol/Cetearyl glucoside/Glucose | 1.20 |
| Stearyl alcohol/Cetyl alcohol | 1.50 |
| Synthetic beeswax | 1.00 |
| Stearic acid | 0.40 |
| Shea butter | 3.00 |
| Caprylic/Capric/Myristic/Stearic triglyceride | 2.00 |
| Macadamia nut oil | 2.00 |
| Hydrogenated poly(C6-14 olefin) | 3.00 |
| Caprylic/Capric triglyceride | 2.00 |
| Sodium acrylate/Sodium acryloyldimethyl taurate | 1.00 |
| copolymer | |
| Ethylhexylglycerin | 0.10 |
| Phenoxyethanol | 0.30 |
| Citric acid | Appropriate |
| | Amount |
| Fragrance | Appropriate |
| | Amount |
| Disodium EDTA | Appropriate |
| | Amount |
| Tocopheryl acetate | Appropriate |
| | Amount |

### Experimental Example 3: Preparation and Evaluation of Composition Including Cell Culture Medium, Albumin, and Solubility Enhancer

### 3-1. Preparation of Composition Including Cell Culture Medium, Albumin, and Solubility Enhancer

A composition was prepared by mixing the SPM disclosed in Preparation Example 1 and various types of solubility enhancers into purified water, as disclosed in the contents in Table 12 below.

TABLE 12

| Example | Component |
|---|---|
| 2 | Purified water (100 g) + SPM (0.78 g) |
| 3 | Purified water (100 g) + SPM (0.78 g) + citric acid |
| | (appropriate amount) |
| 4 | Purified water (80 g) + SPM (0.78 g) + DPG (20 g) |
| 5 | Purified water (100 g) + SPM (0.78 g) + sodium citrate |
| | (appropriate amount) |
| 6 | Purified water (100 g) + SPM (0.78 g) + sodium hydroxide |
| | (appropriate amount) |
| 7 | Purified water (100 g) + SPM (0.78 g) + glycolic acid |
| | (appropriate amount) |

3-2. 3-2.

Evaluation of Solubility The solubility of the Examples 2 to 7 was evaluated.

As a result, it was confirmed that only when citric acid was added as a solubility enhancer, complete dissolution occurred without any residue (Table 13).

TABLE 13

| | | | pH | |
|---|---|---|---|---|
| | | Solubility | Evaluation of | Completely |
| | Examples | Enhancer | Solubility | Dissolved or Not |
| Degree of | 2 | — | 8.58 | X |
| Solubility | | | | |
| Lots of | 3 | Citric acid | 7.56 | ◯ |
| Residue | | | | |
| No | 4 | DPG | 8.65 | X |
| Residue | | | | |
| Lots of | 5 | Sodium | 8.73 | X |
| Residue | | citrate | | |
| Less | 6 | Sodium | 9.47 | X |
| Residue | | hydroxide | | |
| Lots of | 7 | Glycolic | 2.32 | X |
| Residue | | acid | | |

### Experimental Example 4: Preparation and Evaluation of Oil-In-Water Type Cosmetic Composition Including Cell Culture Medium and Albumin

### 4-1. Preparation of Oil-In-Water Type Cosmetic Composition Including Cell Culture Medium and Albumin

To prepare an oil-in-water type cosmetic composition including a cell culture medium and albumin, the following experiment was conducted.

Specifically, according to the content ratios (wt %) shown in Table 14 and Table 15 below, an aqueous phase portion was prepared in a beaker, then stirred at 2,500 rpm at 75° C. for 5 minutes using a Homo Mixer, and then a thickener part was added and stirred at 6,500 rpm for 5 minutes. Next, a dissolved oil phase portion was slowly added to the aqueous phase portion and stirred at 6500 rpm for 5 minutes, after which a neutralizer part was added and stirred at 6500 rpm for 3 minutes. After adding an active component part at 40 to 45° C. through a cooling process, it was mixed at 3500 rpm for 3 minutes and finally cooled to 30° C. to prepare the cosmetic composition.

On the other hand, Table 14 below shows cosmetic compositions prepared according to changes in a thickener, and Table 15 below shows cosmetic compositions prepared according to changes in a surfactant.

TABLE 14

| | Item Name | Example 8 | Example 9 | Example 10 | Example 11 | Example 12 |
|---|---|---|---|---|---|---|
| Aqueous | Purified water | to 100 | to 100 | to 100 | to 100 | to 100 |
| Phase | Glycerin | 5.00 | 5.00 | 5.00 | 5.00 | 5.00 |
| Portion | Xanthan Gum | — | 0.20 | 0.20 | 0.20 | 0.20 |
| | Phenoxyethanol | 0.50 | 0.50 | 0.50 | 0.50 | 0.50 |
| | Hexanediol | 1.50 | 1.50 | 1.50 | 1.50 | 1.50 |
| Thickener | Carbomer | 0.90 | — | — | — | — |
| | Polyacrylate crosspolymer-6 | — | 0.90 | — | — | — |
| | Polyacrylate-13 | — | — | 0.90 | — | — |
| | Sodium acrylate/Sodium | — | — | — | 0.90 | — |
| | acryloyldimethyl taurate copolymer | | | | | |
| | Hydroxyethyl acrylate/Sodium | — | — | — | — | 0.90 |
| | acryloyldimethyl taurate copolymer | | | | | |
| Oil | Glyceryl stearate SE | 3.00 | 3.00 | 3.00 | 3.00 | 3.00 |
| Phase | Cetearyl alcohol | 1.60 | 1.60 | 1.60 | 1.60 | 1.60 |
| Portion | Cetearyl glucoside | 0.40 | 0.40 | 0.40 | 0.40 | 0.40 |
| | Stearic acid | 2.70 | 2.70 | 2.70 | 2.70 | 2.70 |
| | Shea butter | 3.00 | 3.00 | 3.00 | 3.00 | 3.00 |
| | Coconut Oil | 5.00 | 5.00 | 5.00 | 5.00 | 5.00 |
| | Beeswax | 0.50 | 0.50 | 0.50 | 0.50 | 0.50 |
| | Ethylhexyl palmitate | 7.50 | 7.50 | 7.50 | 7.50 | 7.50 |
| | Caprylic/Capric triglyceride | 3.00 | 3.00 | 3.00 | 3.00 | 3.00 |
| | Macadamia nut oil | 1.00 | 1.00 | 1.00 | 1.00 | 1.00 |
| Neutralizer | Purified water | 5.00 | — | — | — | — |
| | Tromethamine | 0.80 | — | — | — | — |
| Active | Purified water | 10.00 | 10.00 | 10.00 | 10.00 | 10.00 |
| Component | Cell culture medium and albumin (SPM) | 0.78 | 0.78 | 0.78 | 0.78 | 0.78 |

TABLE 15

| | Item Name | Example 13 | Example 14 | Example 15 | Example 16 | Example 17 |
|---|---|---|---|---|---|---|
| Aqueous | Purified water | to 100 | to 100 | to 100 | to 100 | to 100 |
| Phase | Glycerin | 5.00 | 5.00 | 5.00 | 5.00 | 5.00 |
| Portion | Xanthan Gum | 0.20 | 0.20 | 0.20 | 0.20 | 0.20 |
| | Phenoxyethanol | 0.50 | 0.50 | 0.50 | 0.50 | 0.50 |
| | Hexanediol | 1.50 | 1.50 | 1.50 | 1.50 | 1.50 |
| Thickener | Polyacrylate crosspolymer-6 | 0.90 | 0.90 | 0.90 | 0.90 | 0.90 |
| Oil | Polysorbate 60 | 1.80 | — | — | — | — |
| Phase | Sorbitan sesquioleate | 0.50 | — | — | — | — |
| Portion | Glyceryl stearate SE | — | 3.00 | — | — | — |
| | Cetearyl glucoside | — | 0.40 | — | — | — |
| | Glyceryl stearate | — | — | 0.75 | — | — |
| | Glyceryl stearate/PEG-100 stearate | — | — | 1.50 | — | — |
| | C14-22 alcohol/C12-20 alkyl glucoside | — | — | — | 0.70 | — |
| | Polyglyceryl-3 methyl glucose distearate | — | — | — | 2.00 | — |
| | Polyglyceryl-6 stearate/Polyglyceryl-6 | — | — | — | — | 0.50 |
| | behenate | | | | | |
| | C12-16 alcohol/Palmitic acid/ | — | — | — | — | 2.00 |
| | Hydrogenated lecithin | | | | | |
| | Cetearyl alcohol | 1.60 | 1.60 | 1.60 | 1.60 | 1.60 |
| | Stearic acid | 2.70 | 2.70 | 2.70 | 2.70 | 2.70 |
| | Shea butter | 3.00 | 3.00 | 3.00 | 3.00 | 3.00 |
| | Coconut Oil | 5.00 | 5.00 | 5.00 | 5.00 | 5.00 |
| | Beeswax | 0.50 | 0.50 | 0.50 | 0.50 | 0.50 |
| | Ethylhexyl palmitate | 7.50 | 7.50 | 7.50 | 7.50 | 7.50 |
| | Caprylic/Capric triglyceride | 3.00 | 3.00 | 3.00 | 3.00 | 3.00 |
| | Macadamia nut oil | 1.00 | 1.00 | 1.00 | 1.00 | 1.00 |
| Active | Purified water | 10.00 | 10.00 | 10.00 | 10.00 | 10.00 |
| Component | Cell culture medium and albumin | 0.78 | 0.78 | 0.78 | 0.78 | 0.78 |

### 4-2. Stability Test of Cosmetic Formulations According to Temperature Changes

To confirm the stability of the oil-in-water type cosmetic formulation of the present disclosure according to temperature change, the following experiment was conducted.

Specifically, the long-term storage stability of the cosmetic formulations of Examples 8 to 17 was evaluated using the evaluation criteria below in a constant temperature bath of 4° C., 45° C., and 50° C., in a cycle (−15° C. to 45° C.), and at room temperature for 1 week, 1 month, and 6 months, and the results are shown in Table 16 and Table 17 below (evaluation criteria, stable: ∘, unstable: Δ, separated: x).

TABLE 16

| Temperature | Duration | Example 8 | Example 9 | Example 10 | Example 11 | Example 12 |
|---|---|---|---|---|---|---|
| 4° C. | 1 Week | ◯ | ◯ | ◯ | ◯ | ◯ |
| | 1 Month | Δ | ◯ | ◯ | ◯ | ◯ |
| | 6 Months | X | ◯ | ◯ | ◯ | ◯ |
| Room | 1 Week | ◯ | ◯ | ◯ | ◯ | ◯ |
| Temperature | 1 Month | Δ | ◯ | ◯ | ◯ | ◯ |
| | 6 Months | X | ◯ | ◯ | ◯ | ◯ |
| 45° C. | 1 Week | Δ | ◯ | ◯ | ◯ | ◯ |
| | 1 Month | X | ◯ | ◯ | ◯ | ◯ |
| | 6 Months | X | ◯ | ◯ | ◯ | ◯ |
| 50° C. | 1 Week | X | ◯ | ◯ | ◯ | ◯ |
| | 1 Month | X | ◯ | ◯ | ◯ | ◯ |
| | 6 Months | — | — | — | — | — |
| Cycle | 1 Week | X | ◯ | ◯ | ◯ | ◯ |
| (−15~45° C.) | 1 Month | X | Δ | Δ | Δ | Δ |
| | 6 Months | — | — | — | — | — |

TABLE 17

| Temperature | Duration | Example 13 | Example 14 | Example 15 | Example 16 | Example 17 |
|---|---|---|---|---|---|---|
| 4° C. | 1 Week | ◯ | ◯ | ◯ | ◯ | ◯ |
| | 1 Month | Δ | ◯ | ◯ | ◯ | ◯ |
| | 6 Months | X | ◯ | ◯ | ◯ | ◯ |
| Room | 1 Week | ◯ | ◯ | ◯ | ◯ | ◯ |
| Temperature | 1 Month | Δ | ◯ | ◯ | ◯ | ◯ |
| | 6 Months | X | ◯ | ◯ | ◯ | ◯ |
| 45° C. | 1 Week | Δ | ◯ | ◯ | ◯ | ◯ |
| | 1 Month | X | ◯ | ◯ | ◯ | ◯ |
| | 6 Months | X | ◯ | ◯ | ◯ | ◯ |
| 50° C. | 1 Week | X | ◯ | ◯ | ◯ | ◯ |
| | 1 Month | X | ◯ | ◯ | ◯ | ◯ |
| | 6 Months | — | — | — | — | — |
| Cycle | 1 Week | X | ◯ | ◯ | ◯ | ◯ |
| (−15~45° C.) | 1 Month | X | Δ | Δ | Δ | Δ |
| | 6 Months | — | — | — | — | — |

As shown in Table 16 and Table 17 above, the formulations of Examples 8 and 13 using polysorbate 60 and sorbitan sesquioleate as surfactants or carbomer as a thickener were unstable, but Examples 9 to 12 and Examples 14 to 17 using different types of surfactants and thickeners were confirmed to have excellent stability. Based on the above results, it may be seen that in the case of the cell culture medium and the oil-in-water type formulation including albumin of the present disclosure, there is no problem with the stability due to the active component of the formulation.

### Experimental Example 5: Preparation and Evaluation of Water-in-Oil Type Cosmetic Composition Including Cell Culture Medium and Albumin

### 5-1. Preparation of Water-in-Oil Type Cosmetic Composition Including Cell Culture Medium and Albumin

To prepare a water-in-oil type cosmetic composition including a cell culture medium and albumin, the following experiment was conducted.

Specifically, according to the content ratios shown in Table 18 and Table 20 below, an oil phase portion was prepared in a beaker, then stirred at 2,500 rpm at 75° C. for 5 minutes using a Homo Mixer to confirm complete dissolution. Next, a dissolved aqueous phase portion was slowly added to the oil phase portion and stirred at 6500 rpm for 5 minutes, and finally cooled to 30 Co to prepare the cosmetic composition.

On the other hand, Table 18 below shows cosmetic compositions prepared by changing a surfactant, Table 19 below shows cosmetic compositions prepared by changing a thickener (stabilizer), and Table 20 below shows cosmetic compositions prepared by changing a wax.

TABLE 18

| | Raw Material Name | Example 18 | Example 19 | Example 20 | Example 21 | Example 22 |
|---|---|---|---|---|---|---|
| Oil | Cyclopentasiloxane | 7.00 | 7.00 | 7.00 | 7.00 | 7.00 |
| Phase | Phenyl trimethicone | 5.00 | 5.00 | 5.00 | 5.00 | 5.00 |
| Portion | PEG-10 dimethicone | — | 3.00 | — | — | — |
| | PEG/PPG-18/18 dimethicone | — | — | 3.00 | — | — |
| | Cetyl PEG/PPG-10/1 dimethicone | — | — | — | 3.00 | — |
| | Bis-PEG/PPG-14/14 dimethicone | — | — | — | — | 3.00 |
| | Hectorite | 1.00 | 1.00 | 1.00 | 1.00 | 1.00 |
| | Beeswax | 1.00 | 1.00 | 1.00 | 1.00 | 1.00 |
| | Dimethicone/Vinyl dimethicone | 0.50 | 0.50 | 0.50 | 0.50 | 0.50 |
| | crosspolymer | | | | | |
| Aqueous | Purified water | to 100 | to 100 | to 100 | to 100 | to 100 |
| Phase | Cell culture medium and albumin | 0.78 | 0.78 | 0.78 | 0.78 | 0.78 |
| Portion | (SPM) | | | | | |
| | Disodium EDTA | 0.02 | 0.02 | 0.02 | 0.02 | 0.02 |
| | Glycerin | 5.00 | 5.00 | 5.00 | 5.00 | 5.00 |
| | Propanediol | 5.00 | 5.00 | 5.00 | 5.00 | 5.00 |
| | Ethylhexylglycerin | 0.05 | 0.05 | 0.05 | 0.05 | 0.05 |
| | 1,2-Hexanediol | 1.00 | 1.00 | 1.00 | 1.00 | 1.00 |
| | Pentylene glycol | 1.50 | 1.50 | 1.50 | 1.50 | 1.50 |

TABLE 19

| | Raw Material Name | Example 23 | Example 24 | Example 25 | Example 26 | Example 27 |
|---|---|---|---|---|---|---|
| Oi | Cyclopentasiloxane | 7.00 | 7.00 | 7.00 | 7.00 | 7.00 |
| Phase | Phenyl trimethicone | 5.00 | 5.00 | 5.00 | 5.00 | 5.00 |
| Portion | PEG-10 dimethicone | 3.00 | 3.00 | 3.00 | 3.00 | 3.00 |
| | Hectorite | — | 1.00 | — | — | — |
| | Quaternium-18 hectorite | — | — | 1.00 | — | — |
| | Stearalkonium hectorite | — | — | — | 1.00 | — |
| | Disteardimonium hectorite | — | — | — | — | 1.00 |
| | Beeswax | 1.00 | 1.00 | 1.00 | 1.00 | 1.00 |
| | Dimethicone/Vinyl dimethicone | 0.50 | 0.50 | 0.50 | 0.50 | 0.50 |
| | crosspolymer | | | | | |
| Aqueous | Purified water | to 100 | to 100 | to 100 | to 100 | to 100 |
| Phase | Cell culture medium and albumin | 0.78 | 0.78 | 0.78 | 0.78 | 0.78 |
| Portion | Disodium EDTA | 0.02 | 0.02 | 0.02 | 0.02 | 0.02 |
| | Glycerin | 5.00 | 5.00 | 5.00 | 5.00 | 5.00 |
| | Propanediol | 5.00 | 5.00 | 5.00 | 5.00 | 5.00 |
| | Ethylhexylglycerin | 0.05 | 0.05 | 0.05 | 0.05 | 0.05 |
| | 1,2-Hexanediol | 1.00 | 1.00 | 1.00 | 1.00 | 1.00 |
| | Pentylene glycol | 1.50 | 1.50 | 1.50 | 1.50 | 1.50 |

TABLE 20

| | Raw Material Name | Example 28 | Example 29 | Example 30 | Example 31 | Example 32 |
|---|---|---|---|---|---|---|
| Oil | Cyclopentasiloxane | 7.00 | 7.00 | 7.00 | 7.00 | 7.00 |
| Phase | Phenyl trimethicone | 5.00 | 5.00 | 5.00 | 5.00 | 5.00 |
| Portion | PEG-10 dimethicone | 3.00 | 3.00 | 3.00 | 3.00 | 3.00 |
| | Hectorite | 1.00 | 1.00 | 1.00 | 1.00 | 1.00 |
| | Beeswax | — | 1.00 | — | — | — |
| | Candelilla wax | — | — | 1.00 | — | — |
| | Carnauba wax | — | — | — | 1.00 | — |
| | Ceresin wax | — | — | — | — | 1.00 |
| | Dimethicone/Vinyl dimethicone | 0.50 | 0.50 | 0.50 | 0.50 | 0.50 |
| | crosspolymer | | | | | |
| Aqueous | Purified water | to 100 | to 100 | to 100 | to 100 | to 100 |
| Phase | Cell culture medium and albumin | 0.78 | 0.78 | 0.78 | 0.78 | 0.78 |
| Portion | Disodium EDTA | 0.02 | 0.02 | 0.02 | 0.02 | 0.02 |
| | Glycerin | 5.00 | 5.00 | 5.00 | 5.00 | 5.00 |
| | Propanediol | 5.00 | 5.00 | 5.00 | 5.00 | 5.00 |
| | Ethylhexylglycerin | 0.05 | 0.05 | 0.05 | 0.05 | 0.05 |
| | 1,2-Hexanediol | 1.00 | 1.00 | 1.00 | 1.00 | 1.00 |
| | Pentylene glycol | 1.50 | 1.50 | 1.50 | 1.50 | 1.50 |

### 5-2. Stability Test of Cosmetic Formulations According to Temperature Changes

To confirm the stability of the water-in-oil type cosmetic formulation of the present disclosure according to temperature change, the following experiment was conducted.

Specifically, the long-term storage stability of the cosmetic formulations of Examples 18 to 32 was evaluated using the evaluation criteria below in a constant temperature bath of 4° C., 45° C. and 50° C., in a cycle (−15° C. to 45° C.) and at room temperature for 1 week, 1 month, and 6 months, and the results are shown in Table 21 to Table 23 below (evaluation criteria, stable: ∘, unstable: Δ, separated: x).

TABLE 21

| Temperature | Duration | Example 18 | Example 19 | Example 20 | Example 21 | Example 22 |
|---|---|---|---|---|---|---|
| 4° C. | 1 Week | X | ◯ | ◯ | ◯ | ◯ |
| | 1 Month | X | ◯ | ◯ | ◯ | ◯ |
| | 6 Months | X | ◯ | ◯ | ◯ | ◯ |
| Room | 1 Week | X | ◯ | ◯ | ◯ | ◯ |
| Temperature | 1 Month | X | ◯ | ◯ | ◯ | ◯ |
| | 6 Months | X | ◯ | ◯ | ◯ | ◯ |
| 45° C. | 1 Week | X | ◯ | ◯ | ◯ | ◯ |
| | 1 Month | X | ◯ | ◯ | ◯ | ◯ |
| | 6 Months | X | ◯ | ◯ | ◯ | ◯ |
| 50° C. | 1 Week | X | ◯ | ◯ | ◯ | ◯ |
| | 1 Month | X | ◯ | ◯ | ◯ | ◯ |
| | 6 Months | — | — | — | — | — |
| Cycle | 1 Week | X | ◯ | ◯ | ◯ | ◯ |
| (−15~45° C.) | 1 Month | X | Δ | Δ | Δ | Δ |
| | 6 Months | — | — | — | — | — |

TABLE 22

| Temperature | Duration | Example 23 | Example 24 | Example 25 | Example 26 | Example 27 |
|---|---|---|---|---|---|---|
| 4° C. | 1 Week | ◯ | ◯ | ◯ | ◯ | ◯ |
| | 1 Month | Δ | ◯ | ◯ | ◯ | ◯ |
| | 6 Months | Δ | ◯ | ◯ | ◯ | ◯ |
| Room | 1 Week | ◯ | ◯ | ◯ | ◯ | ◯ |
| Temperature | 1 Month | Δ | ◯ | ◯ | ◯ | ◯ |
| | 6 Months | Δ | ◯ | ◯ | ◯ | ◯ |
| 45° C. | 1 Week | ◯ | ◯ | ◯ | ◯ | ◯ |
| | 1 Month | Δ | ◯ | ◯ | ◯ | ◯ |
| | 6 Months | Δ | ◯ | ◯ | ◯ | ◯ |
| 50° C. | 1 Week | Δ | ◯ | ◯ | ◯ | ◯ |
| | 1 Month | Δ | ◯ | ◯ | ◯ | ◯ |
| | 6 Months | — | — | — | — | — |
| Cycle | 1 Week | Δ | ◯ | ◯ | ◯ | ◯ |
| (−15~45° C.) | 1 Month | X | Δ | Δ | Δ | Δ |
| | 6 Months | — | — | — | — | — |

TABLE 23

| Temperature | Duration | Example 28 | Example 29 | Example 30 | Example 31 | Example 32 |
|---|---|---|---|---|---|---|
| 4° C. | 1 Week | ◯ | ◯ | ◯ | ◯ | ◯ |
| | 1 Month | ◯ | ◯ | ◯ | ◯ | ◯ |
| | 6 Months | Δ | ◯ | ◯ | ◯ | ◯ |
| Room | 1 Week | ◯ | ◯ | ◯ | ◯ | ◯ |
| Temperature | 1 Month | ◯ | ◯ | ◯ | ◯ | ◯ |
| | 6 Months | Δ | ◯ | ◯ | ◯ | ◯ |
| 45° C. | 1 Week | ◯ | ◯ | ◯ | ◯ | ◯ |
| | 1 Month | Δ | ◯ | ◯ | ◯ | ◯ |
| | 6 Months | Δ | ◯ | ◯ | ◯ | ◯ |
| 50° C. | 1 Week | Δ | ◯ | ◯ | ◯ | ◯ |
| | 1 Month | Δ | ◯ | ◯ | ◯ | ◯ |
| | 6 Months | — | — | — | — | — |
| Cycle | 1 Week | Δ | ◯ | ◯ | ◯ | ◯ |
| (−15~45° C.) | 1 Month | X | Δ | Δ | Δ | Δ |
| | 6 Months | — | — | — | — | — |

As shown in Table 21 to Table 23 above, the formulations of Examples 18, 23 and 28 that did not use a surfactant, thickener (stabilizer), or wax were unstable, but the formulations of Examples 19 to 22, Examples 24 to 27, and Examples 29 to 32 were confirmed to have excellent stability. Based on the above results, it may be seen that in the case of the cell culture medium and the water-in-oil type formulation including albumin of the present disclosure, there is no problem with the stability due to the active component of the formulation.

### Preparation Example 2: Preparation of Composite Powder in which Cell Culture Medium and Albumin are Impregnated in Porous Polymer Powder

### 2-1. Method of Preparing Composite Powder

First, 20 g of powdered cell culture medium and albumin were dissolved in 2000 g of a mixed solvent composed of methanol and dichloromethane in a volume ratio of 1.5:8.5. To the solution in which the active components were dissolved, 15 g of PMMA [Poly(Methyl Methacrylate)] powder, a porous polymer, was added and mixed. The mixed solution was put into a disk-type spray-dryer and spray-dried to produce a composite powder in which the cell culture medium and albumin components were impregnated inside and outside of PMMA. At this time, the spray-drying was performed under the conditions of an air inlet temperature of 95° C., a chamber temperature of 70 to 75° C., a disk rotation speed of 7,500 rpm/min, and a flow rate of 17 kg/h.

### 2-2. Evaluation of Homogenization Level of Composite Powder

To evaluate the homogenization level of the composite powder prepared above, the following experiment was conducted.

Specifically, 300 g of the composition of Preparation Example 1 and the powder of Preparation Example 2 were placed in separate 500 ml containers, shaken 30 times in an up-and-down direction, and then samples were taken from both the upper layer and lower layer to analyze the albumin content.

The content of the mixed composition ratio of Preparation Example 1 was set as 100%, and the analysis was quantitatively performed using HPLC (LC-10AVP, Shimadzu, Japan). The extracted solution was filtered through a syringe filter (pore size 0.2 μm) and then injected, and lysozyme was separated using a VP-ODS column (4.6×250 mm, Shimadzu, Japan). The mobile phase used was a solution of 0.1% trifluoroacetic acid in acetonitrile and 0.1% trifluoroacetic acid in water mixed at a 1:1 ratio, and was measured at a detection wavelength of 220 nm using a UV-detector (SPD-10A, Shimadzu, Japan).

As a result of the above analysis, compared to Preparation Example 1, in the case of Preparation Example 2, in which the cell culture medium and albumin were prepared in the form of a composite powder, there was no difference in the albumin content between the upper layer and lower layer, confirming that the level of homogenization was superior (Table 24).

TABLE 24

| | Preparation | Preparation |
|---|---|---|
| | Example 1 | Example 2 |
| Upper | 35.20% | 99.30% |
| Layer | | |
| Lower | 121.30% | 99.75% |
| Layer | | |

Based on the above results, in the case of the composite powder of Preparation Example 2, it may be seen that the active components are uniformly impregnated into the polymer powder and are homogeneously distributed regardless of the differences in the crystal form, size, and specific gravity of the active components.

### 2-3. Scanning Electron Microscope (SEM) Image

To observe the morphology of the composite powder prepared above, the following experiment was conducted.

Specifically, the composition of Preparation Example 1, the powder of Preparation Example 2, and the porous polymer powder PMMA were observed using a scanning electron microscope (SEM).

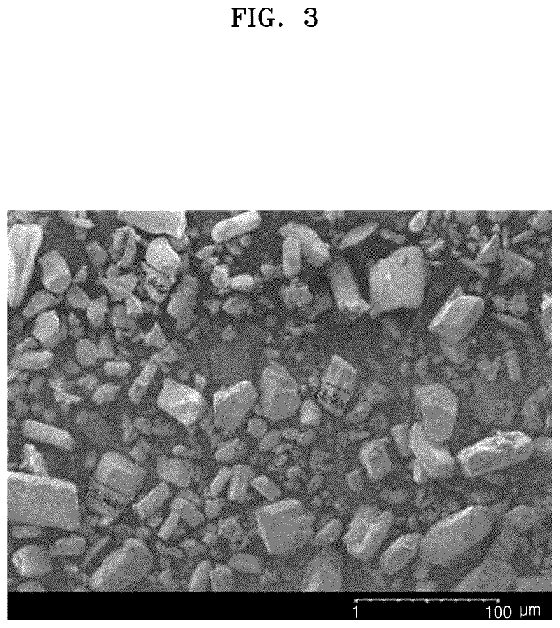

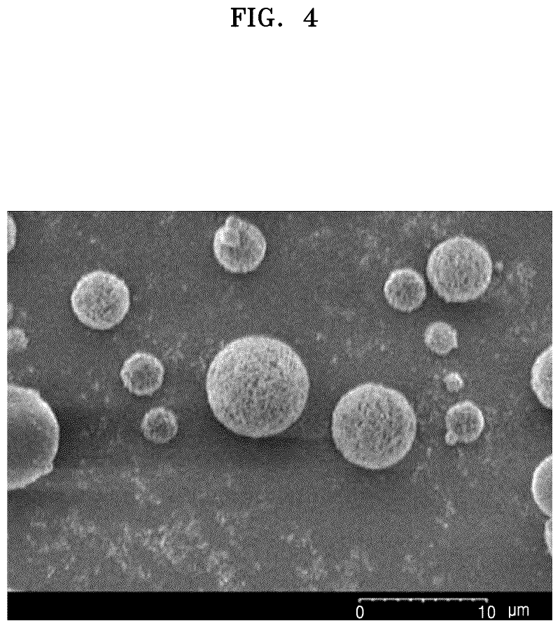

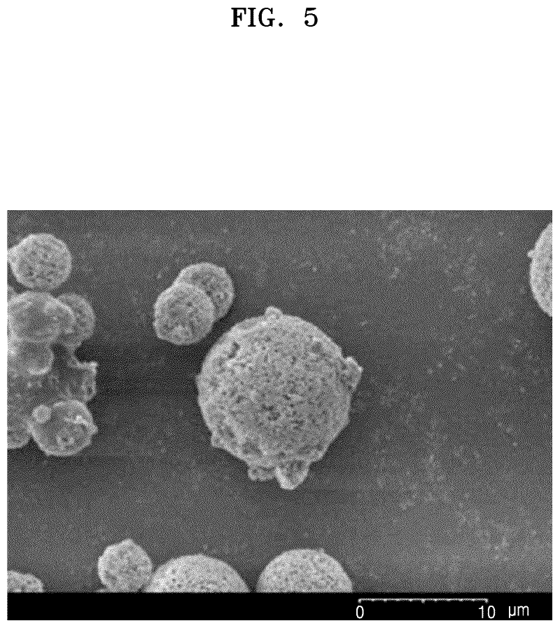

As a result, it was confirmed that active components of various shapes and sizes may be uniformly impregnated into the polymer powder to exhibit a consistent size and shape (FIG. 3 to FIG. 5).

The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the examples described above should be understood in all respects as illustrative and not restrictive.

1-62. (canceled)

63. A cosmetic composition comprising a cell culture medium and albumin as active components.

64. The composition of claim 63, wherein the cell culture medium comprises an amino acid component, a vitamin component, an inorganic salt component, and other components,
wherein the amino acid component comprises, based on 1 part by weight of the amino acid serine, 0.2 to 1.2 parts by weight of glycine, 0.08 to 0.3 parts by weight of L-alanine, 2 to 7 parts by weight of L-arginine hydrochloride, 0.2 to 0.9 parts by weight of L-cysteine hydrochloride-monohydrate, 10 to 18 parts by weight of L-glutamine, 0.8 to 2.0 parts by weight of L-histidine hydrochloride-monohydrate, 1.0 to 5.0 parts by weight of L-lysine hydrochloride, 0.2 to 1.2 parts by weight of L-methionine, 0.2 to 1.2 parts by weight of L-proline, 1 part by weight of L-serine, 1.0 to 5.0 parts by weight of L-threonine, and 1.0 to 5.0 parts by weight of L-valine,
wherein the vitamin component comprises i-inositol, thiamine hydrochloride, niacinamide, and pyridoxine hydrochloride,
wherein the inorganic salt component comprises sodium chloride (NaCl), sodium bicarbonate (NaHCO₃), potassium chloride (KCl), calcium chloride (CaCl₂)) (anhydrous), and sodium dihydrogen phosphate monohydrate (NaH₂PO₄—H₂O),
wherein the other components comprise sodium pyruvate.

65. The composition of claim 64, wherein the vitamin component comprises, based on 1 part by weight of the amino acid serine, 0.2 to 0.9 parts by weight of i-inositol, 0.04 to 0.8 parts by weight of thiamine hydrochloride, 0.04 to 0.8 parts by weight of niacinamide, and 0.04 to 0.8 parts by weight of pyridoxine hydrochloride.

66. The composition of claim 64, wherein the inorganic salt component comprises, based on 1 part by weight of the amino acid serine, 50 to 200 parts by weight of sodium chloride (NaCl), 50 to 120 parts by weight of sodium bicarbonate (NaHCO₃), 2.0 to 20 parts by weight of potassium chloride (KCl), 1.0 to 10 parts by weight of calcium chloride (CaCl₂)) (anhydrous), and 0.5 to 5.0 parts by weight of sodium dihydrogen phosphate monohydrate (NaH₂PO₄—H₂O).

67. The composition of claim 64, wherein the other components comprise, based on 1 part by weight of the amino acid serine, 0.5 to 5.0 parts by weight of sodium pyruvate.

68. The composition of claim 63, wherein the composition further comprises a solubility enhancer.

69. The composition of claim 68, wherein the solubility enhancer comprises a pH adjuster.

70. The composition of claim 68, wherein pH of the composition is 3.0 to 9.0.

71. The composition of claim 69, wherein the pH adjuster comprises one or more selected from the group consisting of citric acid, salicylic acid, glycolic acid, lactic acid, malic acid, tartaric acid, mandelic acid, poly-hydroxy acid (PHA), b-lipohydroxy acid (LHA), oxalic acid, gluconolactone, ascorbic acid, glutamic acid, D-gluconic acid, sulfonic acid, methanesulfonic acid, benzimidazole sulfonic acid, 4-aminobenzoic acid, benzoic acid, sorbic acid, phenylbenzimidazole sulfonic acid, benzylidene camphor sulfonic acid, terephthalylidene dicamphor sulfonic acid, kojic acid, hyaluronic acid, phosphoric acid, carbonic acid, hydrochloric acid, acetic acid, and formic acid.

72. The composition of claim 63, wherein the composition is an oil-in-water (O/W) type or a water-in-oil (W/O) type.

73. The composition of claim 72, wherein the cell culture medium and albumin are in an amount of 0.0001 to 10 wt % based on the total weight of the composition.

74. The composition of claim 72, wherein the composition further comprises one or more selected from the group consisting of surfactants, waxes, butters, thickeners, higher alcohols, and higher fatty acids.

75. The composition of claim 74, wherein the composition is an oil-in-water (O/W) type, and wherein the surfactants comprise one or more selected from the group consisting of glyceryl stearate SE, cetearyl alcohol, cetearyl glucoside, glyceryl stearate, PEG-100 stearate, glyceryl stearate/PEG-100 stearate, C14-22 alcohol, C12-20 alkyl glucoside, C14-22 alcohol/C12-20 alkyl glucoside, polyglyceryl-3 methyl glucose distearate, polyglyceryl-6 stearate, polyglyceryl-6 behenate, polyglyceryl-6 stearate/polyglyceryl-6 behenate, C12-16 alcohol, palmitic acid, hydrogenated lecithin, and C2-16 alcohol/palmitic acid/hydrogenated lecithin.

76. The composition of claim 74, wherein the waxes comprise one or more selected from the group consisting of beeswax, candelilla wax, carnauba wax, multiwax, paraffin wax, microcrystal wax, and ceresin wax.

77. The composition of claim 74, wherein the butters comprise one or more selected from the group consisting of shea butter, mango seed butter, cacao seed butter, cupuacu seed butter, palm seed butter, trichilia emetica seed butter, kokum seed butter, borneo tallow seed butter and sal tree seed butter.

78. The composition of claim 74, wherein the composition is an oil-in-water (O/W) type, and wherein the thickeners comprise one or more selected from the group consisting of guar gum, carrageenan, xanthan gum, carboxymethylcellulose, hydroxyethylcellulose, hydroxypropylcellulose, polyacrylate-13, polyacrylate crosspolymer-6, polyacrylamide, sodium acrylate/sodium acryloyldimethyl taurate copolymer, and hydroxyethyl acrylate/sodium acryloyldimethyl taurate copolymer.

79. The composition of claim 74, wherein the higher alcohols comprise one or more selected from the group consisting of cetyl alcohol, stearyl alcohol, behenyl alcohol, and batyl alcohol.

80. The composition of claim 74, wherein the higher fatty acids comprise one or more selected from the group consisting of stearic acid, isostearic acid, myristic acid, and palmitic acid.

81. The composition of claim 74, wherein the composition is a water-in-oil (W/O) type, and wherein the surfactants comprise one or more selected from the group consisting of PEG-8 dimethicone, PEG-9 polydimethylsiloxyethyl dimethicone, PEG-10 dimethicone, PEG-12 dimethicone, PEG-11 methyl ether dimethicone, cetyl PEG/PPG dimethicone, cetyl PEG/PPG-10/1 dimethicone, bis-PEG/PPG-14/14 dimethicone, bis-PEG/PPG-20/20 dimethicone, PEG/PPG-18/18 dimethicone, PEG/PPG-19/19 dimethicone, PEG/PPG-20/20 dimethicone, polyglyceryl-3 polydimethylsiloxyethyl dimethicone, lauryl polyglyceryl-3 polydimethylsiloxyethyl dimethicone, and lauryl PEG-9 polydimethylsiloxyethyl dimethicone.

82. The composition of claim 74, wherein the composition is a water-in-oil (W/O) type, and wherein the thickeners comprise one or more selected from the group consisting of hectorite, quaternium-18 hectorite, stearalkonium hectorite, and disteardimonium hectorite.

83. A method for improving a skin condition or inflammation in a subject, comprising administering to the subject in need thereof a composition comprising a cell culture medium and albumin as active components.

84. The method of claim 83, wherein improving the skin condition comprises one or more selected from the group consisting of skin moisturization, skin aging improvement, skin barrier strengthening, skin wrinkle improvement, skin elasticity enhancement, skin texture improvement, skin transparency enhancement, skin discoloration and blemish removal, skin whitening, and skin regeneration.

85. The method of claim 83, wherein the inflammation comprises one or more selected from the group consisting of atopic dermatitis, allergic dermatitis, eczema, contact dermatitis, erythema, and psoriasis.

86. A composite powder comprising a cell culture medium, albumin and a porous polymer.

87. The composite powder of claim 86, wherein the cell culture medium comprises an amino acid component, a vitamin component, an inorganic salt component, and other components,
wherein the amino acid component comprises, based on 1 part by weight of the amino acid serine, 0.2 to 1.2 parts by weight of glycine, 0.08 to 0.3 parts by weight of L-alanine, 2 to 7 parts by weight of L-arginine hydrochloride, 0.2 to 0.9 parts by weight of L-cysteine hydrochloride-monohydrate, 10 to 18 parts by weight of L-glutamine, 0.8 to 2.0 parts by weight of L-histidine hydrochloride-monohydrate, 1.0 to 5.0 parts by weight of L-lysine hydrochloride, 0.2 to 1.2 parts by weight of L-methionine, 0.2 to 1.2 parts by weight of L-proline, 1 part by weight of L-serine, 1.0 to 5.0 parts by weight of L-threonine, and 1.0 to 5.0 parts by weight of L-valine,
wherein the vitamin component comprises i-inositol, thiamine hydrochloride, niacinamide, and pyridoxine hydrochloride,
wherein the inorganic salt component comprises sodium chloride (NaCl), sodium bicarbonate (NaHCO₃), potassium chloride (KCl), calcium chloride (CaCl₂)) (anhydrous), and sodium dihydrogen phosphate monohydrate (NaH₂PO₄—H₂O),
wherein the other components comprise sodium pyruvate.

88. The composite powder of claim 87, wherein the vitamin component comprises, based on 1 part by weight of the amino acid serine, 0.2 to 0.9 parts by weight of i-inositol, 0.04 to 0.8 parts by weight of thiamine hydrochloride, 0.04 to 0.8 parts by weight of niacinamide, and 0.04 to 0.8 parts by weight of pyridoxine hydrochloride.

89. The composite powder of claim 87, wherein the inorganic salt component comprises, based on 1 part by weight of the amino acid serine, 50 to 200 parts by weight of sodium chloride (NaCl), 50 to 120 parts by weight of sodium bicarbonate (NaHCO₃), 2.0 to 20 parts by weight of potassium chloride (KCl), 1.0 to 10 parts by weight of calcium chloride (CaCl₂)) (anhydrous), and 0.5 to 5.0 parts by weight of sodium dihydrogen phosphate monohydrate (NaH₂PO₄—H₂O).

90. The composite powder of claim 87, wherein the other components comprise, based on 1 part by weight of the amino acid serine, 0.5 to 5.0 parts by weight of sodium pyruvate.

91. The composite powder of claim 86, wherein the cell culture medium and albumin are impregnated into the porous polymer.

92. The composite powder of claim 86, wherein the porous polymer comprises one or more selected from the group consisting of polymethyl methacrylate [PMMA: poly-(methyl methacrylate)], polyvinyl pyrrolidone [PVP: poly-(vinyl pyrrolidone)], polycaprolactone [PCL: poly-(caprolactone)], polymethylsilsesquioxane [PMSQ: polymethylsilsesquioxane], silica, and poly-L-lactic acid [PLLA: poly-(L-Lactic Acid)].

93. A method of preparing a composite powder comprising a cell culture medium and albumin, comprising:
1) a step of dissolving or dispersing a cell culture medium, albumin and a porous polymer in a solvent; and
2) a step of spray-drying the solution or dispersion obtained above,

94. The preparation method of claim 93, wherein the solvent comprises one or more selected from the group consisting of ethanol, methanol, acetone, and dichloromethane.

95. The preparation method of claim 93, wherein the spray-drying step is conducted by one or more selected from the group consisting of a disk-type spray-dryer and a nozzle-type spray dryer.

96. The preparation method of claim 93, wherein an air inlet temperature of a spray-dryer in the spray-drying step is 80 to 120° C.

97. The preparation method of claim 93, wherein a chamber temperature of a spray-dryer in the spray-drying step is 60 to 90° C.
